(19) United States
(12) Patent Application Publication
(10) Pub. No.: US 2024/0174594 A1
(43) Pub. Date: May 30, 2024

(54) 4-ALKENYL AND 4-ALKYNYL PHENETHYLAMINE DERIVATIVES AND RELATED COMPOUNDS WITH MODIFIED DOM-LIKE ACTION

(71) Applicant: Mind Medicine, Inc., New York, NY (US)

(72) Inventors: Daniel TRACHSEL, Detligen (CH); Matthias Emanuel LIECHTI, Oberwil (CH); Felix LUSTENBERGER, Meggen (CH)

(73) Assignee: Mind Medicine, Inc., New York, NY (US)

(21) Appl. No.: 18/493,385

(22) Filed: Oct. 24, 2023

Related U.S. Application Data
(60) Provisional application No. 63/429,101, filed on Nov. 30, 2022.

(51) Int. Cl. C07C 217/60

(52) U.S. Cl. CPC C07C 217/60

(57) ABSTRACT

A composition of a compound represented by FIGS. 1A-1E for use in substance-assisted therapy. A method of changing neurotransmission, by administering a pharmaceutically effective amount of a compound of FIGS. 1A-1E to a mammal, interacting with serotonin 5-HT2A receptors in the mammal, and inducing psychoactive effects. A method of treating a patient having adverse reactions to psychedelics by administering 4-alkenyl and 4-alkynyl phenethylamine derivatives and related compounds as represented in FIGS. 1A-1E to the patient, and avoiding adverse effects present with psychedelics. A method of changing neurotransmission of an individual, by administering 4-alkenyl and 4-alkynyl phenethylamine derivatives and related compounds as represented in FIGS. 1A-1E and changing neurotransmission in the individual.

### BACKGROUND OF THE INVENTION

### 1. Technical Field

The present invention relates to both, the substance definition and synthesis of 4-alkenyl and 4-alkynyl phenethylamine derivatives and related compounds with modified DOM-like action to be used in substance-assisted psychotherapy.

### 2. Background Art

Psychedelics are substances inducing unique subjective effects including dream-like alterations of consciousness, affective changes, enhanced introspective abilities, visual imagery, pseudo-hallucinations, synesthesia, mystical-type experiences, disembodiment, and ego-dissolution (Liechti, 2017; Passie et al., 2008).

Psychedelics, mainly lysergic acid diethylamide (LSD) and psilocin, are currently investigated as potential medications. First clinical trials indicate potential efficacy of LSD and psilocybin in addiction (Bogenschutz, 2013; Bogenschutz et al., 2015; Johnson et al., 2014; Johnson et al., 2016; Krebs & Johansen, 2012), anxiety associated with life-threatening illness (Gasser et al., 2014; Gasser et al., 2015), depression (Carhart-Harris et al., 2021; Carhart-Harris et al., 2016a; Davis et al., 2021; Griffiths et al., 2016; Roseman et al., 2017; Ross et al., 2016), and anxiety (Griffiths et al., 2016; Grob et al., 2011; Ross et al., 2016). Several trials investigating therapeutic effects of LSD, psilocybin, mescaline, and other psychedelics are also ongoing, as can be seen on www.clinicaltrials.gov. There is also evidence that the psychedelic brew Ayahuasca which contains the active psychedelic substance N,N-dimethyltryptamine (DMT) (Dominguez-Clave et al., 2016) may alleviate depression (Dos Santos et al., 2016; Palhano-Fontes et al., 2019; Sanches et al., 2016). In contrast, there are no comparable therapeutic studies or elaborated concepts on the use of phenethylamines or amphetamines bearing a 2,4,5- or 2,4,6-trisubstitution on the aromatic nucleus such as the psychedelic substance 2,5-dimethoxy-4-methylamphetamine (DOM) or related substances to treat medical conditions.

Although no psychedelic is currently licensed for medical use, psilocybin and LSD are used in special therapeutic-use programs (Schmid et al., 2021). DOM and, although less active and far less investigated, its positional isomer 2,6-dimethoxy-4-methylamphetamine (ψ-DOM), are serotonergic psychedelics similar to LSD and psilocybin with comparable acute effects. DOM or its related compounds can be equally suitable to treat medical conditions. Specifically, existing psychedelic treatments such as LSD, psilocybin, and DMT may not be suitable to be used in all patients considered for psychedelic-assisted therapy. The availability of several substances with different properties is important and the present lack thereof is a therapeutic problem which will further increase with more patients needing psychedelic-assisted therapy and an increase in demand for such treatment once the efficacy of first treatments will be documented in large clinical studies. For example, some patients can react with strong adverse responses to existing therapies such as psilocybin presenting with untoward effects including headaches, nausea/vomiting, anxiety, cardiovascular stimulation, or marked dysphoria. Thus, novel compounds with psychedelic-like action are needed.

Structurally, DOM and W-DOM are alpha-methyl substituted phenethylamines (i.e., aryl-substituted amphetamine derivatives) unlike LSD and psilocybin. LSD, psilocybin, DOM, and 4-DOM are all thought to induce their acute psychedelic effects primarily via their common stimulation of the 5-HT2A receptor. All serotonergic psychedelics including LSD, psilocybin, DMT, mescaline, and DOM are agonists at the 5-HT2A receptor (Rickli et al., 2016) and can therefore produce overall largely similar effects (Snyder et al., 1967). However, there are differences in the receptor activation, receptor subtype selectivity profiles and in the subsequent signal transduction pathway activation patterns between the substances that can induce different subjective effects. LSD potently stimulates the 5-HT2A receptor but also 5-HT2B/C, 5-HT1 and D1-3 receptors. Psilocin, i.e., the active metabolite derived from the prodrug psilocybin in the human body, also stimulates the 5-HT2A receptor but additionally inhibits the 5-HT transporter (SERT). Mescaline binds in a similar, rather low concentration range to 5-HT2A, 5-HT2B, 5-HT2C, 5-HT1A and α2A receptors. DOM shows high affinities at the 5-HT2A and 5-HT2C receptors (Braden & Nichols, 2007; Glennon et al., 1992) but very low affinity at the 5-HT1A receptor (Janowsky et al., 2014)(and data on file). In contrast to LSD, psilocybin and mescaline and DOM show no affinity for D2 receptors (Rickli et al., 2016) (and data on file). Taken together, LSD can have greater dopaminergic activity than psilocybin, mescaline, and DOM, and psilocin (psilocybin) can have additional action at the SERT. DOM and its derivatives do not interact with the SERT in contrast to psilocin (data on file). Taken together, the pharmacological profiles of LSD, psilocybin, and mescaline (as well as DOM) show some differences, but it is not clear whether these are reflected by differences in their psychoactive profiles in humans. This is currently being investigated in a clinical study (www.clinicaltrials.gov: NCT04227756).

In humans, first subjective effects or psychoactive doses of DOM (off of baseline: 1 mg, full active doses: 3-10 mg) appear after 30 minutes, with full effects appearing only after up to 2 hours, and dose-dependently last 14-20 hours (Shulgin & Shulgin, 1991; Snyder et al., 1967).

For the positional isomer ψ-DOM the subjective psychedelic effects have been described in less detail and are observed at an approximately 3- to 5-fold higher dose (15-25 mg) with a significantly shorter duration of action (6-8 hours) (Shulgin & Shulgin, 1991). This difference in potency is quite reliably reflected by 5-HT2A binding data (Chambers et al., 2002; Oberlender et al., 1995; Parker et al., 2008) as well as by rat drug discrimination studies (Chambers et al., 2002; Glennon et al., 1981).

Removal of the a-methyl group of DOM leads to 2,5-dimethoxy-4-methylphenethylamine (2C-D), a compound with significant lower human potency (20-60 mg) and significant shorter duration of action (4-6 hours). The compound 2C-D also showed, at least at these levels of dose, a much lower extent of psychedelic qualities such as ego dissolution, open-eyed visuals, closed-eyes visuals and imagery (Shulgin & Shulgin, 1991).

The acute subjective effects of psychedelics are mostly positive in most humans (Carhart-Harris et al., 2016b; Dolder et al., 2016; Dolder et al., 2017; Holze et al., 2019; Schmid et al., 2015). However, there are also negative subjective effects such as anxiety in many humans likely depending on the dose used, personality traits (set), the setting (physical and social environment) and other factors. The induction of an overall positive acute response to the psychedelic is critical because several studies showed that a more positive experience is predictive of a greater therapeutic long-term effect of the psychedelic (Griffiths et al., 2016; Ross et al., 2016). Even in healthy subjects, a more positive acute response to a psychedelic including LSD has been shown to be linked to more positive long-term effects on well-being (Griffiths et al., 2008; Schmid & Liechti, 2018).

DOM has relevant acute side effects to different degrees depending on the subject treated including increased blood pressure, nausea and vomiting, negative body sensations, and dysphoria. Such side effects of a substance are often linked to its interactions with pharmacological targets. For example, interactions with adrenergic receptors can result untoward clinical cardio-stimulant properties. Additionally, changes in the relative activation profile of serotonin 5-HT receptors and other targets change the quality of the psychoactive effects. Alterations in the binding potency, the binding mode, and the potency in activating the subsequent signaling pathways at 5-HT2A receptors as well as the molecule's lipophilicity can mostly determine the clinical dose to induce psychoactive effects. Alterations changing the metabolic stability of the compounds can also change the duration of action of the substance significantly. DOM as an agent for substance-assisted psychotherapy can be unfavorable in some patients with its unusual slow onset of action of up to 2 hours for full effects, as well as its long duration of action of 14-20 hours. Additionally, such long therapeutic sessions can significantly contribute to the overall therapeutic costs.

New DOM-based derivatives are needed to provide substances with an improved effect profile such as, but not limited to, more positive effects, less adverse effects, different qualitative effects, and shorter duration of acute effect.

### SUMMARY OF THE INVENTION

The present invention provides for a composition of a compound represented by FIGS. 1A-1E for use in substance-assisted therapy, wherein:
R_₁ and R_{┘2} is, independently and in any combination, hydrogen, deuteron, methyl, ethyl, deuterated methyl (D₁-D₃), or deuterated ethyl (D₁-D₅), and
R1, R2 and R3 are, independently and in any combination hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
any halogen; or
nitrogen-containing substituent such as CN or NO₂; and furthermore
R4 is hydrogen, deuteron, and/or fluorine, or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
any halogen; or
a nitrogen-containing substituent such as CN; and furthermore
R5 is fluorine, chlorine, bromine, or iodine; or
SF₅; or
C₂-C₅ branched or unbranched alkyl with the alkyl optionally and independently substituted with F₁-F₁₁ fluorine and/or D₁-D₁₁ deuteron substituents or C₁ alkyl substituted with F₁-F₃ and/or D₁-D₃ deuteron substituents; or
C₃-C₆ cycloalkyl optionally and independently substituted with one or more substituents such as F₁-F₁₅ fluorine and/or D₁-D₁₅ deuteron and/or C₁-C₅ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₅ alkyl; or
a nitrogen-containing substituent such as CN or NO₂; or
C₂-C₅ branched or unbranched alkoxy with the alkoxy optionally and independently substituted with F₁-F₁₁ fluorine and/or D₁-D₁₁ substituents, or C, alkoxy substituted with F₁-F₃ and/or D₁-D₃ deuteron substituents; or
C₃-C₆ cycloalkyloxy optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkoxy optionally and independently substituted with one or more substituents such as F₁-F₁₅ fluorine and/or D₁-D₁₅ and/or C₁-C₅ alkyl; or
C₂-C₅ branched or unbranched alkenyloxy with E or Z vinylic, cis or trans allylic, E or Z allylic or other double bond position in relation to the attached ether function, where any of the carbons of the branched or unbranched alkenyloxy substituent is optionally substituted independently with one or more C₁-C₅ alkyl, and/or with F₁-F₉ fluorine and/or with D₁-D₉ deuteron substituents; or
C₃-C₅ branched or unbranched alkynyloxy, where any of the carbons of the alkynyloxy substituent is optionally substituted independently with one or more C₁-C₅ alkyl, and/or with F₁-F₉ fluorine and/or with D₁-D₉ deuteron substituents; or
C₃-C₅ branched or unbranched alkylthio with the alkylthio group optionally and independently substituted with F₁-F₁₁ fluorine and/or D₁-D₁₁ substituents, or C₁-C₂ alkylthio substituted with F₁-F₅ and/or D₁-D₅ deuteron substituents; or
C₃-C₆ cycloalkylthio optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkylthio optionally and independently substituted with one or more substituents such as F₁-F₁₅ fluorine and/or D₁-D₁₅ and/or C₁-C₅ alkyl; or
C₂-C₅ branched or unbranched alkenylthio with E or Z vinylic, cis or trans allylic, E or Z allylic or other double bond position in relation to the attached thioether function, where any of the carbons of the branched or unbranched alkenylthio substituent is optionally substituted independently with one or more C₁-C₅ alkyl, and/or with F₁-F₉ fluorine and/or with D₁-D₉ deuteron substituents; or
C₃-C₅ branched or unbranched alkynylthio, where any of the carbons of the alkynylthio substituent is optionally substituted independently with one or more C₁-C₅ alkyl, and/or with F₁-F₉ fluorine and/or with D₁-D₉ deuteron substituents.

The present invention provides a method of changing neurotransmission, by administering a pharmaceutically effective amount of a compound of FIGS. 1A-1E to a mammal, interacting with serotonin 5-HT2A receptors in the mammal, and inducing psychoactive effects.

The present invention also provides for a method of treating a patient having adverse reactions to psychedelics by administering 4-alkenyl and 4-alkynyl phenethylamine derivatives and related compounds as represented in FIGS. 1A-1E to the patient, and avoiding adverse effects present with psychedelics.

The present invention also provides for a method of changing neurotransmission of an individual, by administering 4-alkenyl and 4-alkynyl phenethylamine derivatives and related compounds as represented in FIGS. 1A-1E and changing neurotransmission in the individual.

### DESCRIPTION OF THE DRAWINGS

Other advantages of the present invention are readily appreciated as the same becomes better understood by reference to the following detailed description when considered in connection with the accompanying drawings wherein:

FIG. 1A shows the chemical structure of a 4-alkenyl phenethylamine derivative, FIG. 1B shows the chemical structure of a 4-alkenyl phenethylamine derivative, FIG. 1C shows the chemical structure of a 4-alkynyl phenethylamine derivative, FIG. 1D shows the chemical structure of a 4-alkynyl phenethylamine derivative, and FIG. 1E shows the chemical structure of a related compound;

FIGS. 2A-2L show examples of 4-alkenyl and 4-alkynyl phenethylamine derivatives represented by FIGS. 1A-1E within the scope of the invention, FIG. 2A shows compound 12a, FIG. 2B shows compound 12b, FIG. 2C shows compound 12c, FIG. 2D shows compound 12d, FIG. 2E shows compound 12e, FIG. 2F shows compound 12f, FIG. 2G shows compound 16, FIG. 2H shows compound 21, FIG. 2I shows compound 22, FIG. 2J shows compound 18, FIG. 2K shows compound 29, and FIG. 2L shows compound 31;

FIGS. 3A-3J show examples of 4-alkenyl and 4-alkynyl phenethylamine derivatives represented by FIGS. 1A-1E within the scope of the invention, FIG. 3A shows compound 13a, FIG. 3B shows compound 13b, FIG. 3C shows compound 13c, FIG. 3D shows compound 13d, FIG. 3E shows compound 13e, FIG. 3F shows compound 13f, FIG. 3G shows compound 26a, FIG. 3H shows compound 26b, FIG. 3I shows compound 24, and FIG. 3J shows compound 37;

FIGS. 4A-4H show examples of related derivatives represented by FIGS. 1A-1E within the scope of the invention, FIG. 4A shows compound 41, FIG. 4B shows compound 42, FIG. 4C shows compound 55, FIG. 4D shows compound 56, FIG. 4E shows compound 49, FIG. 4F shows compound 50, FIG. 4G shows compound 51, and FIG. 4H shows compound 52;

FIG. 5 summarily describes the synthetic route to the representative compounds 12a-12f and 13a-13f;

FIG. 6 summarily describes the synthetic route to the representative compounds 16, 18, 21 and 22;

FIG. 7 summarily describes the synthetic route to the representative compounds 24, 26a and 26b;

FIG. 8 summarily describes the synthetic route to the representative compounds 29 and 31;

FIG. 9 summarily describes the synthetic route to the representative compound 37;

FIG. 10 summarily describes the synthetic route to the representative compounds 41, 42 and 49-52; and

FIG. 11 summarily describes the synthetic route to the representative compounds 55 and 56.

### DETAILED DESCRIPTION OF THE INVENTION

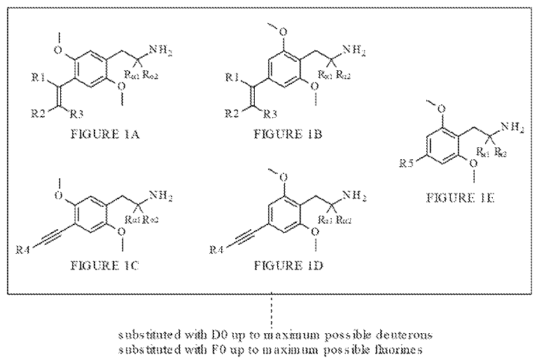

The present invention provides for a composition of 4-alkenyl and 4-alkynyl phenethylamine derivatives and related compounds represented by FIGS. 1A-1E for use in substance-assisted therapy, wherein:
R_₁ and R_{┘2} is, independently and in any combination, hydrogen, deuteron, methyl, ethyl, deuterated methyl (D₁-D₃), or deuterated ethyl (D₁-D₅), and furthermore
R1, R2 and R3 are, independently and in any combination hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
any halogen; or
a nitrogen-containing substituent such as CN or NO₂; and furthermore
R4 is hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN; and furthermore

R5 is fluorine, chlorine, bromine, or iodine; or
SF₅; or
C₂-C₅ branched or unbranched alkyl with the alkyl optionally and independently substituted with F₁-F₁₁ fluorine and/or D₁-D₁₁ deuteron substituents or C₁ alkyl substituted with F₁-F₃ and/or D₁-D₃ deuteron substituents; or
C₃-C₆ cycloalkyl optionally and independently substituted with one or more substituents such as F₁-F₁₅ fluorine and/or D₁-D₁₅ deuteron and/or C₁-C₅ alkyl; or
(C₃-C₆ cycloalkyl)-C₁-C₂ branched or unbranched alkyl optionally and independently substituted with one or more substituents such as F₁-F₁₅ fluorine and/or D₁-Dis deuteron and/or C₁-C₅ alkyl; or
a nitrogen-containing substituent such as CN or NO₂; or
C₂-C₅ branched or unbranched alkoxy with the alkoxy optionally and independently substituted with F₁-F₁₁ fluorine and/or D₁-D₁₁ substituents, or C, alkoxy substituted with F₁-F₃ and/or D₁-D₃ deuteron substituents; or
C₃-C₆ cycloalkyloxy optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkoxy optionally and independently substituted with one or more substituents such as F₁-F₁₅ fluorine and/or D₁-D₁₅ and/or C₁-C₅ alkyl; or
C₂-C₅ branched or unbranched alkenyloxy with E or Z vinylic, cis or trans allylic, E or Z allylic or other double bond position in relation to the attached ether function, where any of the carbons of the branched or unbranched alkenyloxy substituent is optionally substituted independently with one or more C₁-C₅ alkyl, and/or with F₁-F₉ fluorine and/or with D₁-D₉ deuteron substituents; or
C₃-C₅ branched or unbranched alkynyloxy, where any of the carbons of the alkynyloxy substituent is optionally substituted independently with one or more C₁-C₅ alkyl, and/or with F₁-F₉ fluorine and/or with D₁-D₉ deuteron substituents; or
C₃-C₅ branched or unbranched alkylthio with the alkylthio group optionally and independently substituted with F₁-F₁₁ fluorine and/or D₁-D₁₁ substituents, or C₁-C₂ alkylthio substituted with F₁-F₅ and/or D₁-D₅ deuteron substituents; or
C₃-C₆ cycloalkylthio optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkylthio optionally and independently substituted with one or more substituents such as F₁-F₁₅ fluorine and/or D₁-D₁₅ and/or C₁-C₅ alkyl; or
C₂-C₅ branched or unbranched alkenylthio with E or Z vinylic, cis or trans allylic, E or Z allylic or other double bond position in relation to the attached thioether function, where any of the carbons of the branched or unbranched alkenylthio substituent is optionally substituted independently with one or more C₁-C₅ alkyl, and/or with F₁-F₉ fluorine and/or with D₁-D₉ deuteron substituents; or
C₃-C₅ branched or unbranched alkynylthio, where any of the carbons of the alkynylthio substituent is optionally substituted independently with one or more C₁-C₅ alkyl, and/or with F₁-F₉ fluorine and/or with D₁-D₉ deuteron substituents.

In addition to the aforementioned description of compounds represented by FIGS. 1A-1E, any non-protic hydrogen can be replaced by a deuteron or a fluorine in any combination. Specifically, this complements to compounds represented by FIG. 1A-1E by bearing one deuteron up to a completely deuterated compound or bearing one fluorine up to a completely fluorinated compound, with any combination of deuteron and fluorine, and any stereoisomers thereof. More specifically, a deuteration and/or fluorination can be conducted, independently and in any combination, on any of the methoxy groups, on the aromatic nucleus, on any substituent or on the alkylamine sidechain.
R_₁ and R_{┘2} beyond C2, i.e., longer than ethyl, are not recommended as they result in compounds that are much less or even completely pharmacologically not active at all at the target site (5-HT2A receptor).

The compounds represented by FIGS. 1A-1E are basic compounds which form acid addition salts with inorganic or organic acids. Therefore, they form pharmaceutically acceptable inorganic and organic salts with pharmacologically acceptable inorganic or organic acids. Acids to form such salts can be selected from inorganic acids such as hydrochloric acid, hydrobromic acid, hydroiodic acid, sulfuric acid, nitric acid, phosphoric acid, and the like, and organic acids, such as carbonic acid, p-toluenesulfonic acid, methanesulfonic acid, oxalic acid, succinic acid, citric acid, benzoic acid, and the like. Thus, examples of such pharmaceutically acceptable salts are sulfate, pyrosulfate, bisulfate, sulfite, bisulfite, phosphate, monohydrogen-phosphate, dihydrogenphosphate, metaphosphate, pyro-phosphate, chloride, bromide, iodide, formate, acetate, propionate, decanoate, caprylate, acrylate, isobutyrate, caproate, heptanoate, oxalate, malonate, succinate, suberate, sebacate, fumarate, maleate, benzoate, phthalate, sulfonate, phenylacetate, citrate, lactate, glycollate, tartrate, methanesulfonate, propanesulfonate, mandelate and the like. Preferred pharmaceutically acceptable salts are those formed with hydrochloric acid and fumaric acid.

Furthermore, and without loss of generality and elaborating on details, the invention includes any prodrugs, i.e., any chemical modification of the described compounds that is (metabolically) converted to the described compound in the human body. Using a prodrug allows for improving how an active drug is absorbed, distributed, metabolized, and excreted. Prodrugs can be used to prevent release of the active drug in the gastrointestinal tract upon administration so that the drug can be released more favorably elsewhere in the body or to cause an extended, prolonged release. The prodrug can be anything that relies on enzymatic activation and/or that takes advantage of physiological chemical conditions for release of the drug. The basic amino function of the phenethylamine can chemically be transformed to any suitable prodrug that liberates the parent drug. Examples can be, but are not limited to, amide, carbamate, ureate, N-Mannich base, amino sugars, mines (Schiff bases), examines, enaminones, and THTT.

The general chemical terms used for FIGS. 1A-1E have their usual meanings. For example, the term “alkyl” includes such groups as methyl, ethyl, n-propyl, isopropyl, n-butyl, isobutyl, sec-butyl, tert-butyl, and the like. For another example, the term “cycloalkyl” includes such groups as cyclopropyl, cyclobutyl, cyclopentyl, and the like. Further on, the term “alkenyl” includes such groups as vinyl (ethenyl), 1-propenyl, 2-propenyl, isopropenyl, 1-butenyl, 2-butenyl, 3-butenyl and the like. The term “alkynyl” includes groups such as ethynyl, 1-propynyl, 2-propynyl, 1-butynyl, 2-butynyl, 3-butynyl, phenylethynyl, and the like. The term “halogen” includes a fluorine, chlorine, bromine, and iodine substituent and is not limited to any combination or to any number.

Those skilled in the art will appreciate that certain of the compounds of the present invention have at least one chiral carbon, and can therefore exist as a racemate, as individual enantiomers or diastereomers, and as mixtures of individual enantiomers or diastereomers in any ratio. For example, individual enantiomers of compounds of the invention are illustrated in FIG. 1A-1E where R_₁ is different from R_₂. Those skilled in the art will also appreciate that those compounds of the invention where a 4-substituent of a compound in FIG. 1A-1E includes a substituent allowing configurational isomerism, can bear additional isomers. While it is a preferred embodiment of the invention that the compounds of the invention exist are used as racemates or mixtures of diastereomers, the present invention also contemplates the compounds of the invention existing in individual enantiomeric or diastereomeric pure form.

Those skilled in the art will also appreciate that certain of the compounds of the present invention have at least one double bond leading, depending on the double bond's substituents, to cis/trans or E/Z configurational isomerism. While it is a preferred embodiment of the invention that the compounds of the invention exist are used as pure configurational isomers, the present invention also contemplates the compounds of the invention existing in individual cis/trans or E/Z mixtures, respectively.

The individual enantiomers and diastereomers can be prepared by chiral chromatography of the racemic or enantiomerically or diastereomerically enriched amine, or fractional crystallization of salts prepared from racemic- or enantiomerically- or diastereomerically-enriched amine and a chiral acid. Alternatively, the free amine can be reacted with a chiral auxiliary and the enantiomers or diastereomers separated by chromatography or crystallization followed by removal of the chiral auxiliary to regenerate the free amine. Furthermore, separation of enantiomers or diastereomers can be performed at any convenient point in the synthesis of the compounds of the invention. The compounds of the invention can also be prepared by application of chiral syntheses. The compound itself is a pharmacologically acceptable acid addition salt thereof.

The individual cis/trans or E/Z configurational isomers can be accessed by either selective synthesis or by separation techniques addressing the different physicochemical properties of the configurational isomers by applying techniques such as chromatography, crystallization, distillation, or extraction.

In patients that have adverse reactions to other psychedelics, 4-alkenyl and 4-alkynyl phenethylamine derivatives and related compounds can be useful as alternative treatments. In some patients, 4-alkenyl and 4-alkynyl phenethylamine derivatives and related compounds can also be useful because another experience than made with known compounds such as DOM, ψ-DOM, mescaline, psilocybin, or LSD is necessary or because a patient is not suited for therapy with these existing approaches a priori. Thus, 4-alkenyl and 4-alkynyl phenethylamine derivatives and related compounds of subfigures FIG. 1A-E can serve as alternative treatment options with characteristics sufficiently similar to other psychedelics to be therapeutic but also sufficiently different to provide added benefits or avoid negative effects of other psychedelics.

Based on structural relations, the compounds of FIG. 1A-1E described in the present invention are expected to have overall comparable pharmacological properties as DOM and ψ-DOM or other phenethylamine-based psychedelics as described above.

This assumption is further emphasized by the only known and scientific journal-described 4-alkynyl phenethylamine represented by FIG. 1A-1E, a compound named 2C-YN (Trachsel, 2003), which anecdotally has shown psychoactive effects over a short period of 2-3 hours in human (Trachsel et al., 2013).

The present invention provides compounds of FIG. 1A-1E that are pharmacologically active and allow changing the neurotransmission and/or producing neurogenesis. More specifically, but not excluding, the compounds interact with serotonin (5-HT, 5-hydroxytryptamine) 5-HT2A and 5-HT2C receptors in mammals by administering to a mammal in need of such interaction a pharmaceutically effective amount of a compound of FIG. 1A-1E.

Therefore, the present invention provides a method of changing neurotransmission, by administering a pharmaceutically effective amount of a compound of FIG. 1A-1E to a mammal, increasing serotonin 5-HT2A receptor interaction in the mammal, and inducing psychoactive effects.

The neuronal interaction of compounds represented in FIG. 1A-1E can be used in mammals for substance-assisted psychotherapy where the compounds induce psychoactive effect to enhance psychotherapy. The preferred mammal is human.

The intensity and quality of the psychoactive effect including psychedelic or empathogenic effects, the quality of perceptual alterations such as imagery, fantasy and closed or open eyes visuals, and body sensation changes, the pharmacologically active doses, and the duration of action, can be different or similar to that of DOM or ψ-DOM.

DOM and its 4-alkyl homologs as well as its α-desmethyl counterparts (i.e., 2C-D and 4-alkyl homologs) and ψ-DOM are known to interact with serotonin 5-HT2A receptors (Chambers et al., 2002; Nelson et al., 1999).

There have been described numerous 4-substituted 2,5-dimethoxyphenethylamines and 4-substituted 2,5-dimethoxyamphetamines pharmacologically as well as psycho-pharmacologically (Shulgin & Shulgin, 1991; Trachsel et al., 2013). However, a structure-based search with SciFinder revealed that none of the aforementioned compounds contain a 4-alkenyl substituent conjugated with the aryl moiety as shown in subfigure FIG. 1A. Further on, for 4-substituted 2,5-dimethoxyphenethylamines and 4-substituted 2,5-dimethoxyamphetamines there has only been chemically described one compound containing a 4-alkynyl substituent as shown in FIG. 1C, namely 2,5-dimethoxy-4-ethynylphenethylamine (2C-YN).

When it goes to 4-substituted 2,6-dimethoxyphenethylamines and 4-substituted 2,6-dimethoxyamphetamines (called the pseudo series, described with the Greek letter 4J as a prefix), no 4-alkenyl or 4-alkynyl derivative as shown in FIG. 1B and FIG. 1D is known, and there has only been described the 4-substituted 2,6-dimethoxy compounds ψ-2C-O (2,4,6-trimethoxyphenethylamine), ψ-2C-T-4 (2,6-dimethoxy-4-isopropylthiophenethylamine), ψ-DOM (2,6-dimethoxy-4-methylamphetamine) and TMA-6 (2,4,6-trimethoxyamphetamine; could also be called “ψ-TMA-2” but the compound has its own designation). For the latter four compounds, human psychoactivity has only been manifested for ψ-DOM and TMA-6 so far (Shulgin & Shulgin, 1991). For the compound ψ-DOM there has been shown an approximately three-fold lower 5-HT2A binding affinity in comparison to its positional isomer DOM (Chambers et al., 2002; Oberlender et al., 1995; Parker et al., 2008). Further on, its ability to substitute for known psychedelics in rat drug discrimination studies has been presented (Chambers et al., 2002; Glennon et al., 1981). There has also been described qualitatively the chemical access to a series of ψ-2C-O (4-alkoxy-2,6-dimethoxyphenethylamines) and ψ-MRM (4-alkoxy-2,6-dimethoxyamphetamines) derivatives, but no binding data has been given (Trachsel et al., 2013). The last hitherto identifiable ψ-compound, ψ-2C-B, has been described chemically as an intermediate for the preparation of some PGE2 receptor modulators (Patent: WO2018210994). In summary, while hundreds of 2,4,5-trisubstituted phenethylamines and amphetamines are known, only a handful of 2,4,6-trisubstituted derivatives are described. The reason for this may lie in the chemical access of such compounds which needs much more efforts in comparison to the 2,4,5-trisubstituted derivatives.

Based on data available and on structure-activity relationships the invented 4-alkenyl and 4-alkynyl phenethylamine derivatives and related compounds presented in FIG. 1A-1E are assumed to behave as 5-HT2A receptor ligands and to be psychoactive with psychedelic properties.

Some of the invented 4-alkenyl and 4-alkynyl phenethylamine derivatives and related compounds represented by FIG. 1A-1E have shown high affinities at the aforementioned receptors (Trachsel, Hoener, Liechti, personal communication, data on file), comparable or higher as DOM and ψ-DOM and indicating psychedelic action.

Not only the nature of psychedelic experiment, receptor interactions and subsequent signaling pathways can change by structural modifications represented in FIG. 1A-1E but also parts of ADME (absorption, distribution, metabolism and excretion), e.g., the metabolism can be modified significantly by making, as an example, but is not limited to, a potentially labile alkenyl or alkynyl compound more or less prone to metabolism by introducing alkyl groups, aryl or heteroaryl groups, nitriles, fluorine atoms and deuterium atoms to these functional groups in either vinyl, allyl or gamma positions, or in ethynyl or propargyl positions as aforementioned. Thus, the invention allows also for the synthesis of psychedelic compounds with a relatively shorter duration of action compared to more metabolically stable and longer-acting compounds.

The structure of 4-alkenyl and 4-alkynyl phenethylamine derivatives represented in FIG. 1A-1D was previously only described for one single compound in a scientific journal (Trachsel, 2003), namely the compound 2,5-dimethoxy-4-ethynylphenethylamine (2C-YN). Only the chemical preparation was presented, not the pharmacological profiles and human therapeutic uses.

The structure of 4-substituted 2,6-dimethoxyphenethylamine derivatives as represented in FIG. 1E has only been described for 4-methoxy, 4-bromo, 4-isopropylthio, and 4-methyl analogs, but no human therapeutic profiles are mentioned.

4-alkenyl and 4-alkynyl phenethylamine derivatives can include 2,5- and 2,6-dimethoxy substitution variations of the phenethylamine structure forming “2C-enes” and “2C-ynes”, respectively, as well as the pseudo (ψ)-analogs “ψ-2C-enes” or “ψ-2C-ynes”, or can include the addition of a methyl group to the alpha carbon of the amino function of the corresponding phenethylamine structures to form amphetamines also containing the above 2,5- or 2,6-dimethoxy substituents on the phenyl ring to form “DO-enes” or “DO-ynes”, respectively, as well as the pseudo (ψ)-analogs “ψ-DO-enes” or “ψ-DO-ynes” (Shulgin & Shulgin, 1991; Trachsel et al., 2013). Further on, the aforementioned alpha carbon to the amino function (shown as R_₁ and R_₂ in subfigures FIG. 1A-E) can also be substituted, independently and in any combination, with one or two deuterons, methyl, ethyl, deuterated methyl (D₁-D₃), or deuterated ethyl (D₁-D₅) groups. Further on, any number of deuterons and independently and in any combination, any number of fluorine substituents can be contained in the structures shown in FIG. 1A-1D. Several new such 4-alkenyl and 4-alkynyl phenethylamine derivatives represented in FIG. 1A-1D were newly synthesized in the present invention.

4-substituted 2,6-dimethoxyphenethylamine derivatives as represented in FIG. 1E can include substituents such as an alkyl, cycloalkyl, alkoxy, cycloalkoxy, cycloalkylalkoxy, alkylthio, cycloalkylthio, cycloalkylalkylthio, halogen, nitrile, nitro, aryl, and heteroaryl. The alpha carbon to the amino function can be substituted (R_₁ and R_₂), independently and in any combination, with one or two deuterons, methyl, ethyl, deuterated methyl (D₁-D₃), or deuterated ethyl (D₁-D₅) groups. Further on, any number of deuterons, and independently and in any combination, any number of fluorine substituents can be contained in the structures shown in FIG. 1E. Several new such 4-substituted phenethylamine derivatives represented in FIG. 1E were also newly synthesized in the present invention.

While all the 4-alkenyl and 4-alkynyl phenethylamine derivatives and related compounds represented in FIG. 1A-1E are useful in optimizing the clinical effect profile of psychedelics, certain classes of the compounds are preferred, such as wherein the compound is a free base, a salt, a hydrochloride salt, a racemate where applicable, a single enantiomer, a single diastereomer, or a mixture of enantiomers or diastereomers in any ratio, or an individual of a cis/trans or E/Z configurational isomer, or a mixture of these configurational isomers in any ratio. It will be understood that these classes can be combined to form additional preferred classes.

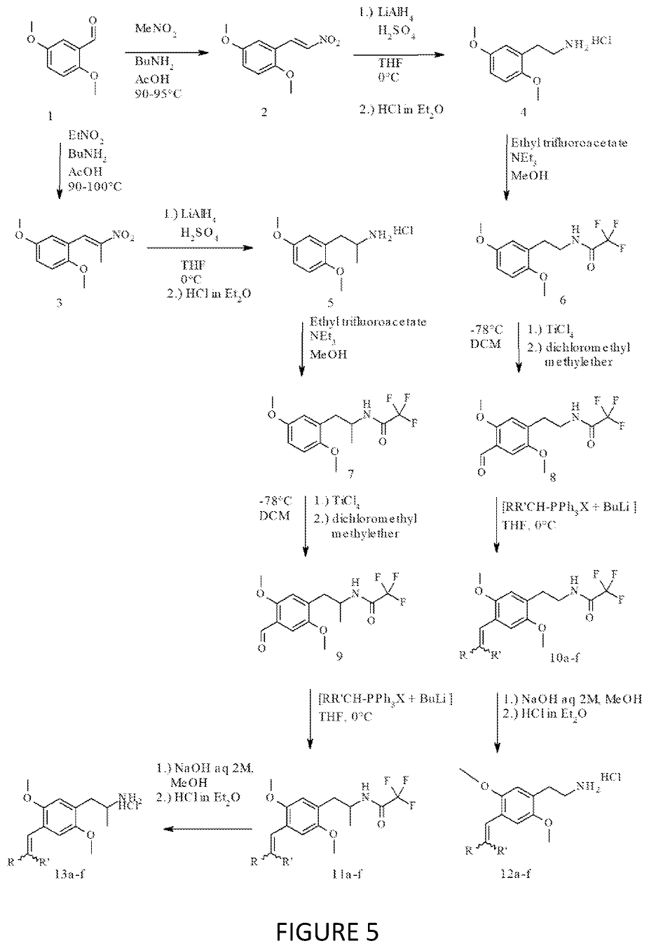

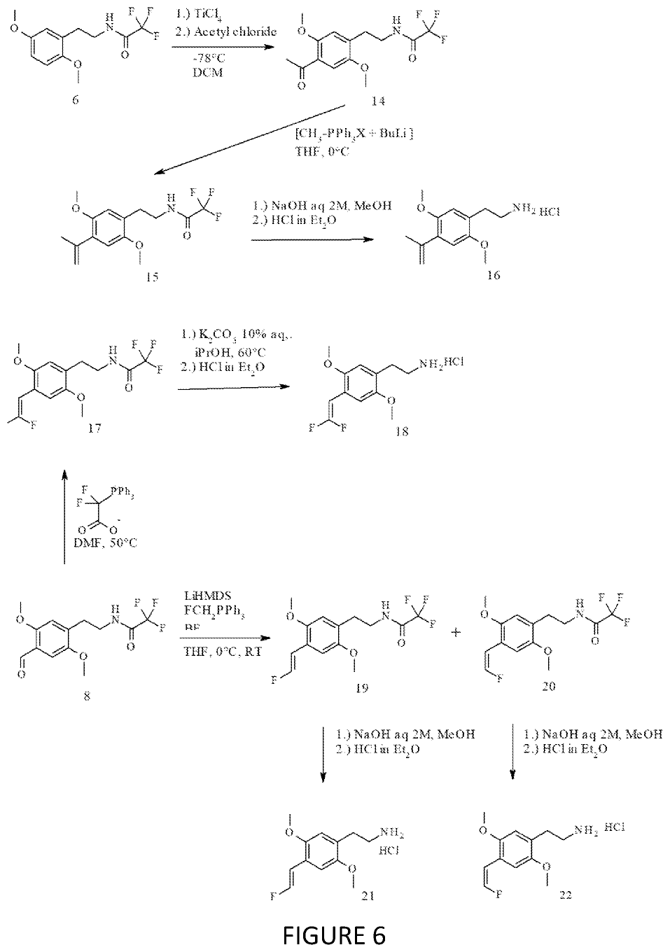

The synthetic access to 4-substituted 2,5-dimethoxyphenethylamines and their amphetamine counterparts is often achieved by first introducing the 4-substituent into 2,5-dimethoxybenzaldehyde, then condensing it with a nitroalkane to the corresponding nitroolefin, which is finally reduced with, e.g., lithium aluminum hydride to the final compound. The preparation of the nitroolefins from these benzaldehydes was achieved by the reaction with nitromethane or nitroethane, generally referred as the Henry reaction, using, e.g., n-butylamine and acetic acid as well as molecular sieves, an advantageous catalytic system described earlier by the inventors (provisional patent application entitled: “mescaline derivatives with modified action,” U.S. 63/153,317, filing date 2/24/2021). The nitroolefins are reduced to the corresponding desoxyscalines or 3C-desoxyscalines by using lithium aluminum hydride (LAH) or alane generated in situ from LAH and concentrated sulfuric acid, in analogy to earlier works (Shulgin & Shulgin, 1991; Trachsel, 2002). Due to chemical and economic reasons the inventors chose somewhat different approach: the synthetic access described before is not always suitable for accessing compounds represented in FIGS. 1A-1E containing a 4-unsaturated substituents or substituents labile to the conditions mentioned above. Thus, additional routes were set up, and a parallel synthesis approach that allows a structural diversification late in the synthesis was chosen, reducing synthetic efforts (FIG. 5 and FIG. 6). Following this, for accessing compounds represented by subfigure FIG. 1A, N-trifluoroacetyl protected 2,5-dimethoxyphenethylamine as well as N-trifluoroacetyl protected 2,5-dimethoxyamphetamine were prepared. They could be converted to the corresponding 4-formyl derivatives by using TiCl₄ and dichloromethyl methyl ether (Märcher-Rorsted et al., 2021) As valuable building blocks they were then allowed to react with Wittig ylides or Wittig-type reagents to form 4-aryl-conjugated alkenes. Any of the N-trifluoroacetyl protected products accessed by the routes mentioned before could then be N-deprotected in order to access final compounds represented in FIG. 1A. In a similar way, other carbonyl groups can be introduced, allowing further access to 4-alkenyl derivatives being branched at the vinyl-C attached to the arene moiety (FIG. 6).

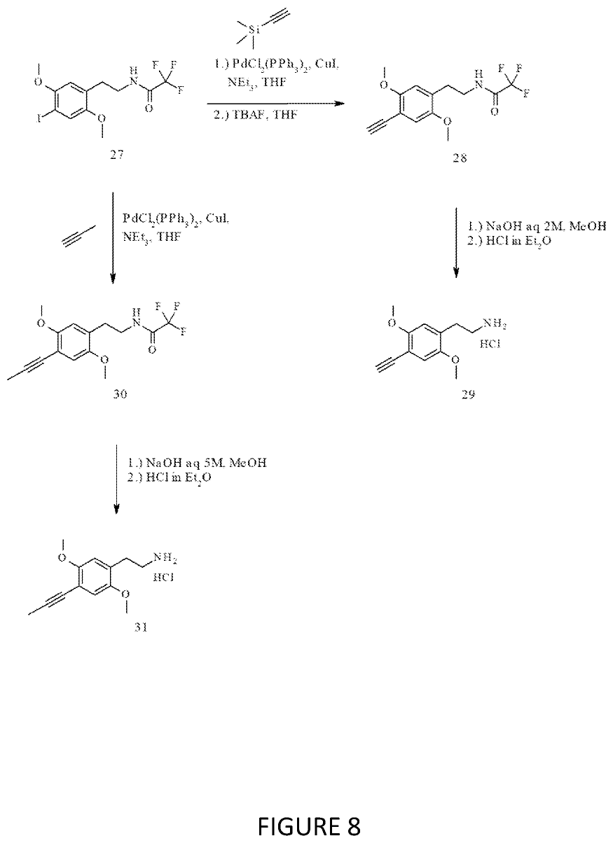

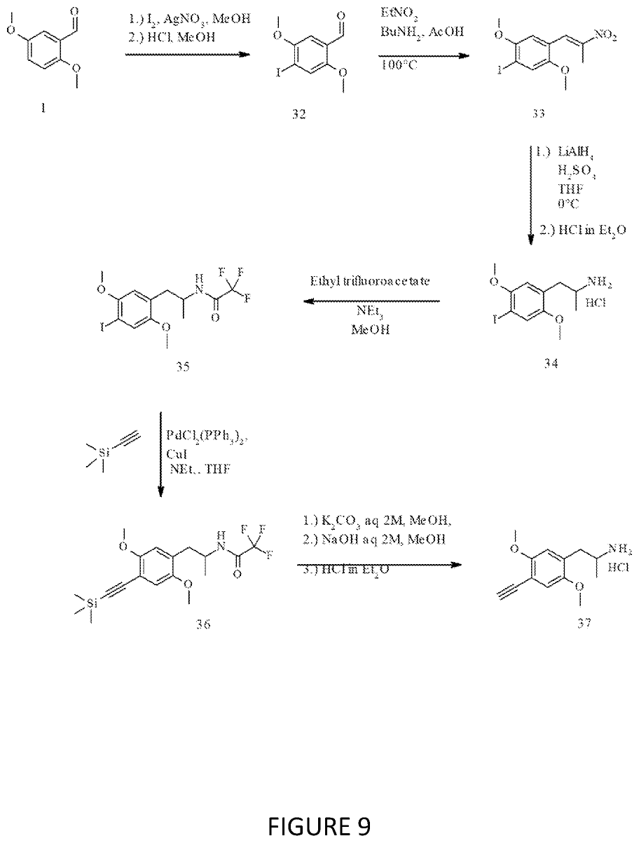

Access to 4-alkynyl-2,5-dimethoxy derivatives as represented in FIG. 1C was achieved by using the known N-trifluoroacetyl protected 2,5-dimethoxy-4-iodophenethylamine as well as N-trifluoroacetyl protected 2,5-dimethoxy-4-iodoamphetamine and allowing them to react with the corresponding alkynes in a Sonogashira-type reaction (FIGS. 8 and 9). The N-trifluoroacetyl protected products accessed by these routes could then be N-deprotected in order to access final compounds represented in FIG. 1C.

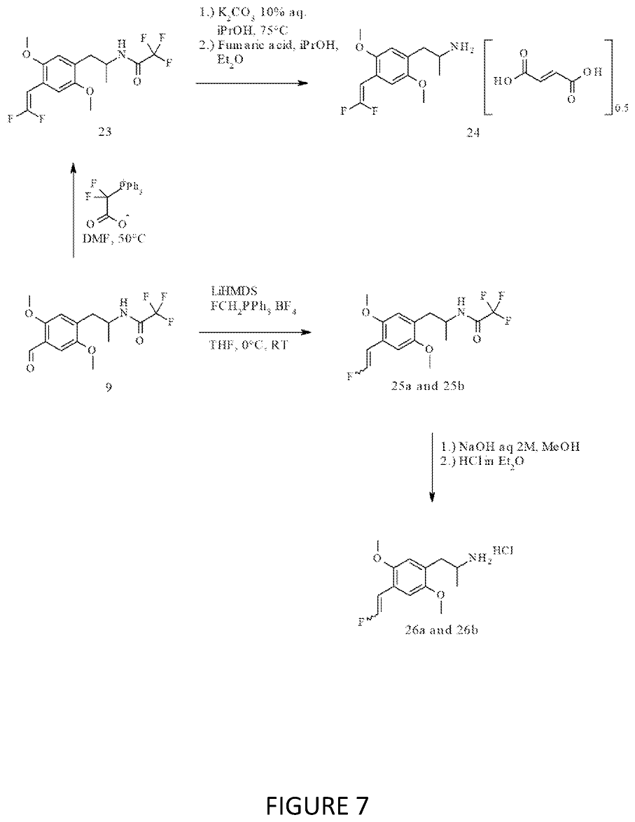

Generally, fluorinated vinyl groups are hitherto rarely used in medicinal chemistry and remain completely unexplored among the psychoactive phenethylamines and amphetamines, and the inventors are the first exploring this scaffold (Trachsel, Liechti and Lustenberger; U.S. 63/153,317, mescaline derivatives with modified action). In a similar way to the above Wittig reactions, vinyl-fluorinated compounds can be accessed from N-trifluoroacetyl protected 2,5-dimethoxy-4-formylphenethylamine as well as N-trifluoroacetyl protected 2,5-dimethoxy-4-formylamphetamine by using corresponding Wittig-type reagents (FIG. 6 and FIG. 7). The N-trifluoroacetyl protected products accessed by these routes can then be N-deprotected in order to access final compounds represented in FIG. 1A.

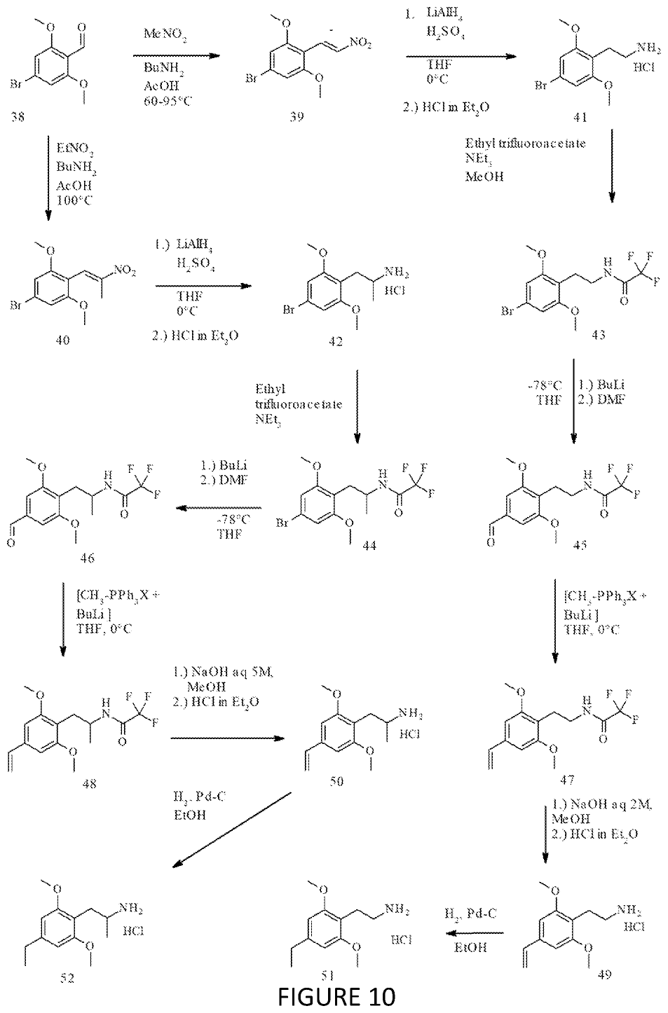

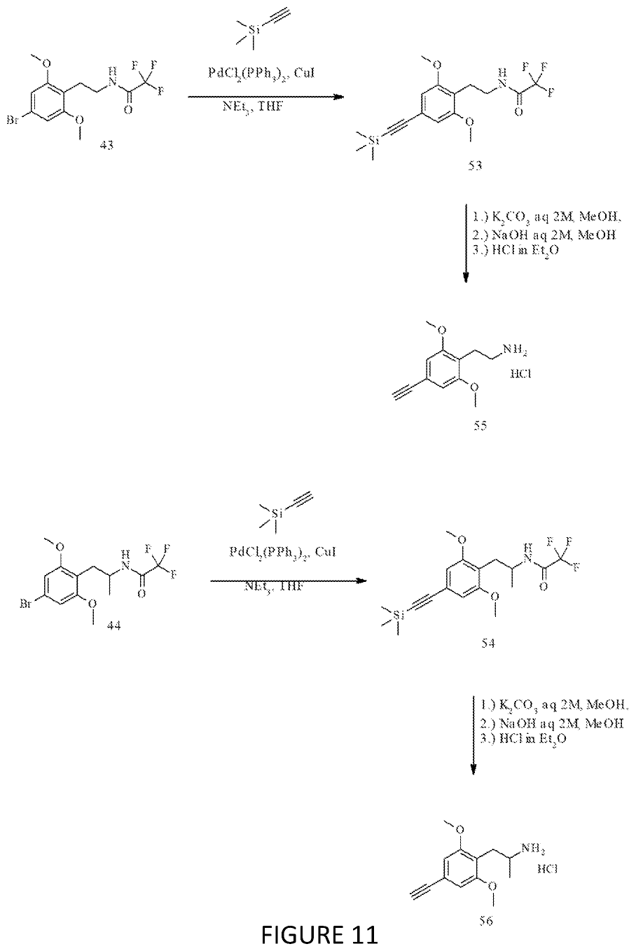

Due to chemical reasons (e.g., electronic pi and sigma donating/attracting as well as steric and coordinating effects), compounds with a 2,4,6-trisubstitution as represented in FIGS. 1B, 1D, and 1E are generally only accessible with much greater chemical efforts, one of the main reasons of their extremely low representation within the phenethylamine psychedelics. To access such compounds represented in FIGS. 1B, 1D, and 1E, a slightly different access to the corresponding 4-alkenyl-2,6-dimethoxyphenethylamines and amphetamines was used, in that the 4-formyl group was introduced from the corresponding 4-bromo derivative (FIG. 10). The chemical 4-bromo-2,6-dimethoxybenzaldehyde, a compound now commercially available, was converted to N-trifluoroacetyl protected 4-bromo-2,6-dimethoxyphenethylamine as well as to N-trifluoroacetyl protected 4-bromo-2,6-dimethoxyamphetamine. These valuable building blocks were then further allowed to react in Wittig-type and Sonogashira reactions (FIGS. 10 and 11) and finally N-deprotected in order to access 4-alkenyl and 4-alkynyl 2,6-dimethoxy derivatives as represented in FIGS. 1B and 1D.

Any of the unsaturated 4-substituent introduced as described before can also be reduced by classical conditions such as the use of hydrogen and a catalyst such as palladium on activated charcoal to access the corresponding 4-alkylated derivatives, as exemplarily outlined in FIG. 10, to access compounds represented in FIG. 1E.

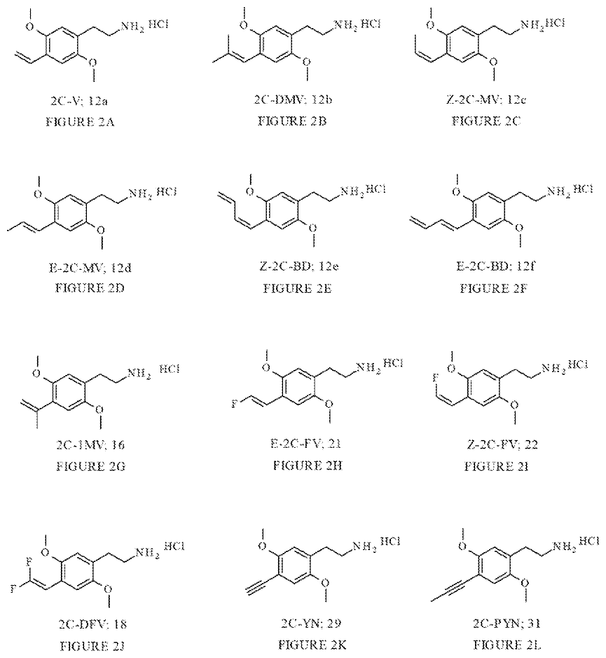

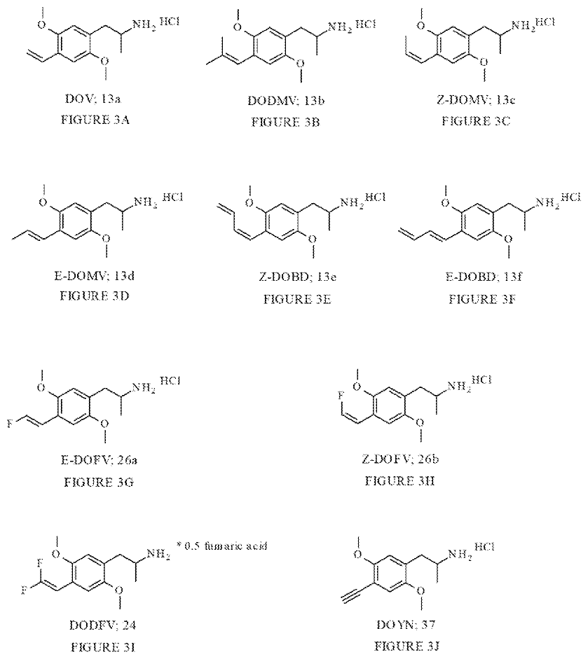

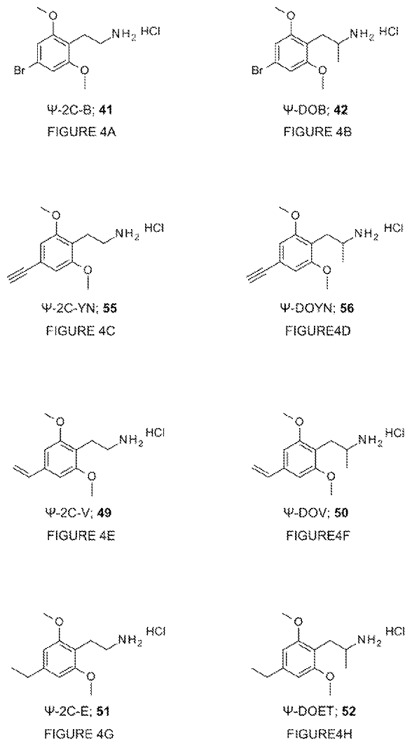

The group presented in the preparation section, namely compounds 12a-12f, 13a-13f, 16, 18, 21, 22, 24, 26a-26b, 29, 31, 37, 41, 42, 49, 50, 51, 52, 55 and 56 (chemical structures see FIGS. 2A-4H), is illustrative of 4-alkenyl and 4-alkynyl phenethylamine derivatives and related compounds represented in FIG. 1A-1E contemplated within the scope of the invention.

A small selection of the synthesized 4-alkenyl and 4-alkynyl phenethylamines and their amphetamine congeners and related compounds as shown in FIG. 1A-1E were investigated at the key target for psychoactive effects in vitro (Trachsel, Hoener, Liechti, personal communication, data on file). The main target of psychedelics is the 5-HT2A receptor (Holze et al., 2021) and typically there is a high affinity binding at this receptor (Rickli et al., 2016). Additionally, the binding potency at the 5-HT2A receptor is typically predictive of the human doses of psychedelics to be psychoactive for many compounds (Luethi & Liechti, 2018). Furthermore, the psychedelic effects of psilocybin in humans have been shown to correlate with 5-HT2A receptor occupancy measures using positron emission tomography (Madsen et al., 2019). Thus, interactions with this target are relevant and predict psychedelic action with high likelihood for most psychedelics. However, this may not be the case for all substances within this class.

Additional receptors such as the serotonergic 5-HT1A and 5-HT2C or dopaminergic D2 receptors are thought to moderate the effects of psychedelics (Rickli et al., 2016). Although some psychedelics like psilocybin do not directly act on dopaminergic receptors, they have nevertheless some dopaminergic properties by releasing dopamine in the striatum (Vollenweider et al., 1999) likely via 5-HT1A receptor activation (Ichikawa & Meltzer, 2000). Furthermore, LSD has activity at D2 receptors (Rickli et al., 2016) and some of its behavioral effect may be linked to this target (Marona-Lewicka et al., 2005).

Activity of compounds at monoamine transporters are thought to mediate MDMA-like empathogenic effects (Hysek et al., 2012). Importantly, mescaline is a very weak 5-HT2A receptor ligand and high doses are needed to induce psychoactive effects in humans. However, despite its low potency, mescaline can have extraordinarily strong psychedelic effects in humans at high doses.

The preliminary serotonin 5-HT2A binding data (Trachsel, Hoener, Liechti, personal communication, data on file) suggests compounds from FIG. 1A-1E being psychedelic active, since they bear 4-substituents with comparable steric bulkiness and lipophilicity and bind in the same order of magnitude or even with higher affinity as comparative psychedelic compounds such as DOM and ψ-DOM (Shulgin & Shulgin, 1991), to be evaluated in detail clinically. Key results of the preliminary pharmacological profiling of the compounds described herein were:

Some of the 4-alkenyl and 4-alkynyl phenethylamine derivatives and related compounds represented in FIG. 1A-1E showed relevant binding affinity and intrinsic activity at the serotonin 5-HT2A receptor indicating activity as psychedelics. For some compounds, the binding affinity was superior to DOM.

Together, the in vitro profiles of 4-alkenyl and 4-alkynyl phenethylamine derivatives and related compounds represented in FIG. 1A-1E compared with that of psilocybin, LSD, DOM, and ψ-DOM indicate overall psychedelic properties when used in humans. Accordingly, some 4-alkenyl and 4-alkynyl phenethylamine derivatives and related compounds can exert psychedelic acute effect profiles that are more beneficial to some patients including but not limited to: more overall positive effects, more or less perceptual effects, more emotional effects, less anxiety, less cardio-stimulant effects, less adverse effects, less nausea, longer and also shorter effects among other properties and compared to DOM.

There are several problems when using known psychedelics such as LSD and DOM that can be solved using the compounds described herein. Namely, long therapeutic session durations (LSD: 8-12 hours, DOM 14-20 hours) are needed due to the long duration of psychoactive effects, and thus either therapists need to care their patients for an unreasonable long time—with that being significant economic disadvantages —, or psychedelic trips need to be stopped by administering an antidote such as a serotonin 5-HT2 antagonist (U.S. Ser. No. 17/156,233) or a benzodiazepine, which may, in certain cases, be disadvantageous. The presently developed substances represented in FIG. 1A-1E were designed to have similar qualitative effects to DOM while acting shorter or to have a comparable duration of action but other qualitative effects as reflected by their structural changes and associated pharmacological properties. In particular, some metabolically less-stable compounds were created to shorten the plasma half-life and duration of action in humans. Other alterations of the chemical structure were designed to create substances with qualitative effects different from those of DOM and creating subjective effects that are considered beneficial to assist psychotherapy including feelings of empathy, openness, trust, insight, and connectedness and known to those knowledgeable in the field. Further problems when using known psychedelics like psilocybin can rise from producing adverse effects including nausea and vomiting, cardiovascular stimulation, and an increase in body temperature and others. The novel compounds can produce less nausea, less cardio stimulation, less thermogenesis and/or other adverse responses.

The compounds represented by FIG. 1A-1E act with shorter or with comparable duration of action in human in comparison to the original DOM molecule. This is triggered by modification of the molecular structure in FIG. 1A-1E.

The present invention therefore also provides for a method of treating a patient having adverse reactions to psychedelics by administering a 4-alkenyl or 4-alkynyl phenethylamine derivative or related compounds to the patient, and avoiding adverse effects present with psychedelics.

The group presented in the preparation section, namely compounds 12a-12f, 13a-13f, 16, 18, 21, 22, 24, 26a-26b, 29, 31, 37, 41, 42, 49, 50, 51, 52, 55 and 56 (chemical structures see FIGS. 2A-4H), is illustrative of 4-alkenyl and 4-alkynyl phenethylamine derivatives and related compounds represented in FIGS. 1A-1E contemplated within the scope of the invention.

The compounds according to the invention and represented in FIGS. 1A-1E allow modification of the duration and mode of action, the psychodynamic processes, and the qualitative perceptions, e.g., in terms of psychedelic or empathogenic intensity in comparison to the original DOM molecule.

The compounds according to the invention and represented in FIGS. 1A-1E can cause similar or different quality of imagery, fantasy and closed or open eyes visuals in comparison to the original DOM molecule.

The compounds according to the invention and represented in FIGS. 1A-1E can have a lower, a similar or a higher dose potency in comparison to the original DOM molecule. The dose of the compounds can be dosed according to known doses for DOM described above, off of baseline: 1 mg, full active doses: 3-10 mg.

The compounds according to the invention and represented in FIGS. 1A-1E can cause similar or more favorable body feelings in comparison to the original DOM molecule.

The aforementioned characteristics can be additionally modified in a progressive way by the introduction of one or more fluorine atoms, by one or more deuterium atoms and by one or more alkyl groups, independently as well as in any combination, to the 4-alkenyl or 4-alkynyl group or generally to the 4-substituent in any position of these groups or substituents.

The aforementioned characteristics can further be modified in a progressive way by the introduction of one or more fluorine atoms, by one or more deuterium atoms, by one or more alkyl group, and by one or more functional group such as carbon-, nitrogen-, oxygen-, or sulfur-containing, independently as well as in any combination, to any position of the compounds represented in FIGS. 1A-1E, namely to the 4-substituent, to any of the methoxy groups, to the aromatic nucleus, to the alkylamine side chain or to any other introduced substituent on the compounds of invention.

This is outlined in more detail by the following relationships. The overall chemical/biological stability of a compound of invention as represented in FIGS. 1A-1E towards acidic, basic or any other chemical/biological conditions is dependent on factors such as pH, solvent medium, temperature, surface, nucleophilicity or electrophilicity of reaction partners or on gas containment of the environment. Metabolic stability in a biological environment such as a human body is additionally driven by factors such absorption rate, exposure to enzymes, enzyme activity, genetic polymorphism, retention time in a body medium such as gastrointestinal tract, rate of body distribution or transportation times. All these aspects can be influenced by the structural changes introduced to the compounds and result in the desired effects and effect-durations in humans.

The stability of a functional group, a substituent or, generally spoken, a molecule, towards aforementioned factors can significantly be influenced and modified by specific incorporation of stabilizing or destabilizing atoms or atom groups. Furthermore, the overall metabolic stability of a compound is also driven by properties such as the overall lipophilicity, three-dimensional structure, dissociation constants, solubility, steric accessibilities and steric bulkiness and other characteristics.

Fluorine is a strong electron-withdrawing atom and its incorporation to a substituent can significantly reduce the electron richness. Further on, it modifies dipole moment, dissociation constants of acidic and basic groups, the lipophilicity, pH value, and, to a certain extent, also steric properties of a fluorine-containing molecule are influenced. Thus, fluorine can chance physicochemical properties and incorporation into a molecule can have a dramatic influence on interaction with biological targets, on chemical/metabolic stabilities and on metabolic pathways. Fluorine atoms incorporated to a molecule further allows so-called multipolar interactions with partially charged functional groups. This makes fluorine as an excellent tool for medicinal chemistry.

Deuteron is a stable isotope of hydrogen. Due to its slightly different metric, incorporated into a molecule it can influence physicochemical properties. With this, kinetic isotope effects, inverse kinetic isotope effects and steric isotope effects can also be observed. Chemical bonds involving deuterium are stronger and of different length compared to protium (hydrogen), which make such compounds significantly different in biological reactions. Thus, incorporation of a deuteron into a molecule can greatly influence its biological stabilities.

Consequently, both fluorine and deuteron can be used to replace or to be added to a substituent to modify the overall stability and biological properties of the compounds invented and represented by FIGS. 1A-1E. Replacement can be done with one or more fluorine atoms or with one or more deuteron atoms or in any combination of fluorine and deuteron atoms.

In analogy to these medicinal chemistry concepts, the biological properties of the invented compounds (ADME, target selectivity and target interaction, the mode of action, duration of action, the psychodynamic processes, and the qualitative perceptions, e.g., in terms of psychedelic or empathogenic intensity in comparison to the original DOM molecule) can not only be influenced by the aforementioned application of fluorine or deuteron to the functional groups such as the 4-alkenyl or 4-alkynyl groups or generally spoken to the 4-substituent but also to any position of the phenethylamine core structure of the compounds in FIGS. 1A-1E. Other atoms or atom groups can be used in a similar way to all these positions.

Taken together, the modified properties can be tailored and applied individually to the patient's need. This is not only targeted by changing the compound's receptor profile but also greatly by the modification of ADME (Absorption, Distribution, Metabolism and Excretion) via the scope of invention outlined before in compounds represented in FIGS. 1A-1E.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, subcutaneously, or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

### Preparation of the Compounds

A general access to the 4-alkenyl and 4-alkynyl phenethylamine derivatives and related compounds represented in FIG. 1A-1E is outlined in FIGS. 5 to 11.

Commercially available 2,5-dimethoxybenzaldehyde (1) is subjected to an aldol condensation, namely the Henry reaction, by mixing the aldehyde with a nitroalkane such as nitromethane, nitroethane or 1-nitropropane and a catalyst such as an organic salt or a mixture of an organic base and an organic acid, most favorably n-butylamine and acetic acid (such as illustrated in FIG. 5). The mixture can or not then be treated with heat in absence or presence of a drying agent such as an inorganic salt or, most favorably, molecular sieves. The water formed may also be removed azeotropically during reaction. The reaction mixture is cooled, and the product solids formed are filtered of, or the mixture is concentrated in vacuo prior further treatment. The obtained residue is further purified by crystallization or recrystallization or by column chromatography in order to get the final nitroolefins such as 2 or 3 (FIG. 5) as well as such as 33, starting from 2,5-dimethoxy-4-iodobenzaldehyde (FIG. 9) and, similarly, such as 39 and 40, starting from 4-bromo-2,6-dimethoxybenzaldehyde (FIG. 10).

As continuation of the illustrative invention, the obtained nitroalkenes are dissolved in an inert solvent such as tetrahydrofuran or diethyl ether and added to a suspension of alane generated in situ from allowing to react lithium aluminum hydride (LiAlH₄) with concentrated sulfuric acid (H₂SO₄) in a similar solvent (such as illustrated in FIG. 5, FIG. 9, and FIG. 10). The reaction temperature is set between −20° C. and 70° C., favorably at 0° C.-60° C. The reduction can also be performed by any other suitable conditions, such as the use of LiAlH₄ without H₂SO₄. The reaction mixture is then quenched subsequently with an alcohol, favorably isopropanol, and then with a base such as aqueous sodium hydroxide before filtering it off. The Filtrate is concentrated in vacuo and during the process an inert gas such as argon or nitrogen can be applied in order to prevent any carbamate formation. The residual amine free base (such as of structure 4-5 and 34 as well as of 41-42, as illustrated in FIG. 5, FIG. 9, and FIG. 10) is then dissolved in a solvent, favorably non-protic, most favorably in diethyl ether or dioxane, and neutralized by the addition of anhydrous hydrogen chloride or sulfuric acid or any other salt forming organic agent such as fumaric acid, tartaric acid, or acetic acid in a similar solvent.

To access compounds represented in FIG. 1A, a synthetic pathway can be followed, as illustrated in FIG. 5. The compounds 12a-f and 13a-f are prepared from the corresponding 2,5-dimethoxy-4-formylphenethylamine or its amphetamine counterpart bearing a suitable protecting group on the basic nitrogen. Such protecting groups can be N-trifluoroacetyl or any other conditions-resistant group. As such, templates 8 and 9 were prepared starting from the amines 4 and 5 and the trifuoroacetylating agent ethyl trifluoroacetate in MeOH and NEt₃, but any other trifuoroacetylating agent, solvent and base can be used. The N-trifluoroacetyl protected compounds 6 and 7 where then 4-formylated using TiCl₄ and dichloromethyl methylether in dichloromethane from a known procedure (MArcher-Rorsted et al., 2021) to access 8 and 9. As illustrated in FIG. 5, the obtained 4-formyl derivatives 8 and 9 can then be used for C-C coupling reactions to form 4-alkene-containing compounds. As illustrative examples, a Wittig-type reaction can be applied, e.g., by the use of a Wittig salt, which is first converted to a suitable ylide by the use of, as illustrated in FIG. 5, a base such as butyllithium, KOtBu, or LiHMDS in a suitable organic inert solvent such as THF, dioxane or diethyl ether at a reaction temperature allowing the reaction to be sufficiently selective. More specifically, a temperature of −80° C. to 20° C. and more favorably at −20° C. to 10° C. is applied. The 4-formyl derivatives, such as 8 and 9, are then added to the ylides prepared as described before. Depending on the reaction conditions and reagents and additives used, one can force the reaction to yield a preferred amount of either of the configurational alkene isomers E or Zor cis or trans. Alternatively, the configurational isomers can also be separated by any suitable technique such as chromatography, crystallization, or distillation. To access the final compounds the protecting group is removed by known procedures. Illustrative, for N-trifluoroacetyl protected compounds (10a-f and 11-f, FIG. 5), a suitable base can be aqueous sodium hydroxide in methanol, but any other suitable conditions can be applied. The residual free base (such as of 12a-f and 13a-f, as illustrated in FIG. 5) is then converted to a suitable salt by conditions as described above.

To access compounds represented in FIG. 1A that are branched at the vinyl-C attached to the aromatic nucleus and as shown with compound 16 in FIG. 6, the dichloromethyl methylether from the above reaction can be replaced by any other acylating agent such as acetyl chloride. By such, carbonyl intermediate 14 was obtained. This intermediate can serve to access many other compounds represented by FIG. 1A, and as an example, was converted to the final derivative 16 by applying a Wittig reaction, N-deprotection and salt conversion as outlined before.

Compounds representing examples of FIG. 1A containing 4-fluorinated vinyl groups, such as compounds 18 or 24 can be accessed by the illustrative reactions of N-trifluoroacetyl-2,5-dimethoxy-4-formylphenethylamine (8, FIG. 6) or N-trifluoroacetyl-2,5-dimethoxy-4-formylamphetamine (9, FIG. 7) with (triphenylphosphonio)difluoroacetate in an organic solvent such as DMF or DMSO at elevated temperatures, favorably at 0-100° C., and more favorably at 40-50° C. to form the difluorovinyl intermediates 17 (FIG. 6) and 23 (FIG. 7) which can be N-deprotected with a suitable base or a reagent. A favored base can be aqueous potassium carbonate in isopropanol and using elevated temperatures such as 40-100° C., but any other suitable conditions can be applied to get the corresponding free bases of compounds 18 and 24. Final salt conversion can be performed as outlined before, and for compounds such 18 and 24 fumarate salt formation is preferred.

Furthermore, compounds representing examples of FIG. 1A containing 4-fluorinated vinyl groups, such as compounds 21 and 22 or 26a and 26b can be accessed by the illustrative reactions of N-trifluoroacetyl-2,5-dimethoxy-4-formylphenethylamine (8, FIG. 6) or N-trifluoroacetyl-2,5-dimethoxy-4-formylamphetamine (9, FIG. 7) in a Wittig reaction with fluoromethyl-(triphenylphosphonium) tetrafluoroborate or any other suitable Wittig-type reagent in the Wittig reaction as outlined above. The configurational (double bond) isomers of the corresponding fluorovinyl intermediates 19, 20 or 25a and 25b can be separated and then N-deprotected and converted to their salts 21, 22 and 26a and 26b by methods outlined above.

Access to compounds such as 4-alkynyl-2,5-dimethoxyphenethylamines (29 and 31, FIG. 8) and amphetamines (37, FIG. 9) represented by FIG. 1C was achieved by using the known N-trifluoroacetyl protected 2,5-dimethoxy-4-iodophenethylamine (27, FIG. 8) as well as N-trifluoroacetyl protected 2,5-dimethoxy-4-iodoamphetamine (35, FIG. 9) and allowing them to react with the corresponding alkynes in a Sonogashira-type reaction (FIGS. 8 and 9). The reaction was conducted at ambient temperature using Pd(PPh₃)₂Cl₂ as catalyst, NEt₃ as base and THF as solvent, but any other catalytic, base, solvent and temperature system suitable for successful conversion can be applied. The N-trifluoroacetyl protected products accessed by these routes (compounds 28, 30 and 36) could then be acetylene-deprotected, where necessary, by using TBAF in THF or a base such as aqueous K₂CO₃ in MeOH, and N-deprotected and converted to their salts as outlined before in order to access final compounds such as 29 and 31 (FIG. 8) and 37 (FIG. 9) and as represented in subfigure FIG. 1C.

Compounds represented in FIGS. 1B, 1D, and 1E consist of a 2,4,6-trisubstitution on the aromatic nucleus, and compounds bearing a 4-alkenyl substituent as represented in FIG. 1B are exemplified by compounds 49 and 50 (FIG. 10). They can be accessed in a similar way as was outlined for compounds of FIG. 1A represented by chemical synthesis of compounds 12a-f and 13a-f, with some differences, by starting from commercially available 4-bromo-2,4-dimethoxybenzaldehyde (38) instead: Henry reaction to access the nitroolefins 39 and 40 (FIG. 10), reduction to the amines 41 and 42, N-protecting to the N-trifluoroacetates 43 and 44, which can then be converted to the 4-formyl analogs 45 and 46 by suitable conditions. As illustrated in FIG. 10, these conditions can consist of a metalation step using a suitable agent such as butyllithium in a suitable organic inert solvent such as THF, dioxane or diethyl ether, at a reaction temperature of −150° C. to −0° C., more favorably at −120° C. to −70° C. Next, the metalated intermediates are treated with a formyl-introducing agent such as dimethylformamide. Compounds 45 and 46 are then converted in a Wittig reaction as outlined before to the corresponding alkenes, with compounds 47 and 48 being representatives. They are finally N-deprotected and converted to their salts, as outlined before, to access representative compounds of FIG. 1B, namely 49 and 50.

Access to compounds such as 4-alkynyl-2,6-dimethoxyphenethylamines (55, FIG. 11) and amphetamines (56, FIG. 11) represented by FIG. 1D was achieved by using the above N-trifluoroacetyl protected 2,6-dimethoxy-4-bromophenethylamine (43, FIG. 11) as well as N-trifluoroacetyl protected 2,6-dimethoxy-4-bromoamphetamine (44, FIG. 11) and subsequently allowing them to react with the corresponding alkynes in a Sonogashira-type reaction, followed by acetylene and N-deprotections and finally conversion to their salts, as outlined in detail above.

Compounds represented by the FIG. 1E are exemplified by compounds 41, 42, 51 and 52 (FIG. 10). Access to 41 and 42 is discussed above; these two compounds not only serve as chemical intermediates but also as compounds within the scope of invention. To access compounds wherein R5 (FIG. 1E) consists of an alkyl substituent, any of the unsaturated 4-substituent introduced as described before could also be reduced by classical conditions such as the use of hydrogen and a catalyst such as palladium on activated charcoal in a solvent such as ethanol to access the corresponding 4-alkylated derivatives, as exemplarily outlined in FIG. 10, starting from the hydrochloride salts 49 and 50, respectively, to easily access compounds 51 and 52, representing examples of FIG. 1E.

### Detailed Description of the Chemical Preparation of the Compounds

General. NMR was performed on a Bruker NMR (¹H: 300 MHz and ¹⁹F: 282 MHz) at ambient temperature. Reaction controls were performed by silica gel TLC (F₂₅₄; UV detection) and HPLC UV DAD & MS (Agilent 1100, Waters SQD).

General method for the nitro olefination (modified Henry reaction). The aryl aldehyde is dissolved in nitromethane or nitroethane under slight warming. Next, molecular sieves 3 Å (where applied), n-butylamine and acetic acid is added, and the mixture is gently stirred at 60-110° C. under an inert atmosphere. When the reaction is complete (monitoring by TLC, eluent, e.g.: dichloromethane) the mixture is separated from the molecular sieves (where applied) and concentrated in vacuo. The residue is either recrystallized from an appropriate solvent or purified by dissolving it in a small amount of organic solvent and eluting it with organic solvent through a short-path silica gel column. The eluate obtained is concentrated in vacuo. Usually, the trans-nitroolefin is obtained. In some cases, minor amounts of the cis-nitroolefin were observed as well; upon reduction (see next step) both cis and trans isomers lead to the same products and thus no separation is required.

General method for the alane-promoted reduction of the nitroolefins. To an ice-cooled suspension of lithium aluminum hydride (LiAlH₄) in tetrahydrofuran (THF) anhydr. is added dropwise sulfuric acid (H₂SO₄) 95-99% under an inert atmosphere and vigorous stirring. When hydrogen evolution has ceased the mixture is stirred for another 5-10 minutes. Next, a solution of the nitroolefin in THF anhydr. is added under ice-cooling at such a rate that the reaction becomes not too violent, and the reaction temperature stays below 20-30° C. After completion of addition the mixture is brought to a gentle reflux for 3-5 minutes, and then again cooled with an ice-bath. Next, the mixture is cautiously quenched by successive and dropwise addition of anhydr. isopropanol (IPA) and then aqueous 2M sodium hydroxide solution (NaOH). Occasionally, THF is added to keep the mixture stirrable. When hydrolysis is complete, the mixture is filtered off and the filter cake is rinsed well with THF. The filtrate is concentrated in vacuo; purging the apparatus can be performed by applying an inert gas such as nitrogen or argon which prevents the formation of any unwanted carbamates.

General method for the hydrochloride salt formations. The amine base is dissolved in approx. 30-50 times the mass of anhydr. diethyl ether containing 0.5% anhydr. IPA. The well stirred solution is cautiously neutralized by the addition of 2M anhydr. HCl in diethyl ether or 4M anhydr. HCl in dioxane and occasional cooling; the pH should not be far from neutral in order to not get a sticky mass during processing. The suspension obtained is filtered off, rinsed with diethyl ether, and dried in vacuo to get the final hydrochloride product.

### Examples—Preparation of the 4-alkenyl-2,5-dimethoxyphenethylamine derivatives 12a-f, 13a-f, 16, 18, 21, 22, 24 and 26a-b

### 1.) Preparation of N-TFA Protected Phenethylamine Templates 6 and 7

2,5-Dimethoxy-β-nitrostyrene, 2. According to the general method for the nitro olefination described, from 50 g 2,5-dimethoxybenzaldehyde (1), 100 mL nitromethane, 2 mL butylamine and 2 mL acetic acid, 50 minutes at 90° C. Yield: 51 g (81%) 2 as orange crystals. ¹H-NMR (CDCl₃): 3.82 (s, MeO), 3.93 (s, MeO), 6.93 (d, 1 arom. H), 6.98 (d, 1 arom. H), 7.04 (dxd, 1 arom. H), 7.86 (d, CHNO₂), 8.13 (d, CH═CHNO₂).

1-(2,5-Dimethoxyphenyl)-2-nitropropene, 3. According to the general method described for the nitro olefination, from 50 g 2,5-dimethoxybenzaldehyde (1), 120 mL nitroethane, 2 mL butylamine, 2 mL acetic acid and 20 g mol sieves, 4 hours at 90° C. Yield: 52.5 g (78%) 3 as orange crystals. ¹H-NMR (CDCl₃): 2.41 (d, MeC), 3.81 (s, MeO), 3.85 (s, MeO), 6.87 (d, 1 arom. H), 6.89 (d, 1 arom. H), 6.97 (dxd, 1 arom. H), 8.25 (s, CH═C).

2,5-Dimethoxyphenethylamine hydrochloride (2C—H), 4. According to the general method described for the alane-promoted nitro olefin reduction, from 9.0 g 2, 6.08 g LiAlH₄, 4.25 mL H₂SO₄, 130 mL plus 110 mL THF, 25.3 mL IPA and 19.4 mL NaOH 2M. Hydrochloride salt formation according to the general method described. Yield: 7.80 g (83.3%) product 4 as a white solid. ¹H-NMR (D₂O): 2.94 (t, ArCH₂), 3.21 (t, CH₂NH₃_{⁺}), 3.78 (s, MeO), 3.80 (s, MeO), 6.87 (d, 1 arom. H), 6.92 (dxd, 1 arom. H), 7.01 (d, 1 arom. H).

2,5-Dimethoxyamphetamine hydrochloride (2,5-DMA), 5. According to the general method described for the alane-promoted nitro olefin reduction, from 9.4 g 3, 6.08 g LiAlH₄, 4.25 mL H₂SO₄, 130 mL plus 110 mL THF, 25.3 mL IPA and 19.4 mL NaOH 2M. Hydrochloride salt formation according to the general method described. Yield: 6.96 g (71.3%) product 5 as a white solid. ¹H-NMR (D₂O): 1.23 (d, MeCH), 2.07 (s, ArCH₃), 2.88 (d, ArCH₂), 3.62 (m, CHNH₃_{⁺}), 3.77 (s, MeO), 3.79 (s, MeO), 6.85 (d, 1 arom. H), 6.88 (dxd, 1 arom. H), 7.01 (d, 1 arom. H).

N-Trifluoroacetyl-2,5-dimethoxyphenethylamine, 6. To a solution of 7.80 g (35.8 mmol) 4 and 10.97 mL (78.76 mmol; 2.2 eq) NEt₃ in 60 mL MeOH anhydr. was added dropwise 4.25 mL (39.38 mmol; 1.1 eq) ethyl trifluoroacetate within 2 minutes under nitrogen. After stirring for 45 minutes at ambient temperature, the volatiles were removed in vacuo and the residue was redissolved in MTBE (150 mL), washed with aq. HCl 0.5M (2×100 mL) and water (2×100 mL), dried over Na₂SO₄ and concentrated in vacuo to get 8.84 g (89.1%) product 6 as an orangish-beige solid. ¹H-NMR (CDCl₃): 2.91 (t, ArCH₂), 3.58 (q, CH₂NH), 3.78 (s, MeO), 3.83 (s, MeO), 6.34 (bs, NH), 6.73 (d, 1 arom. H), 6.79 (dxd, 1 arom. H), 6.85 (d, 1 arom. H), 7.09 (bs, NH).

N-Trifluoroacetyl-2,5-dimethoxyamphetamine, 7. To a solution of 6.86 g (29.6 mmol) 5 and 9.07 mL (65.1 mmol; 2.2 eq) NEt₃ in 50 mL MeOH anhydr. was added dropwise 3.51 mL (32.56 mmol; 1.1 eq) ethyl trifluoroacetate within 2 minutes under nitrogen. After stirring for 90 minutes at ambient temperature, the volatiles were removed in vacuo and the residue was redissolved in MTBE (130 mL), washed with aq. HCl 0.5M (2×80 mL) and water (2×80 mL), dried over Na₂SO₄ and concentrated in vacuo to get 7.49 g (86.9%) product 7 as a beige solid. ¹H-NMR (CDCl₃) revelated an approx. 80:20 mixture of cis/trans amide mixture (isomers not assigned; major product peak's shifts given): 1.28 (d, MeCH), 2.86 (m, ArCH₂), 3.79 (s, MeO), 3.85 (s, MeO), 4.15 (m, CHNH), 6.72 (d, 1 arom. H), 6.79 (dxd, 1 arom. H), 6.85 (d, 1 arom. H), 7.43 (bs, NH).

### 2.) Introduction of the Carbonyl Group: Preparation of Templates 8, 9 and 14

N-Trifluoroacetyl-2,5-dimethoxy-4-formylphenethylamine, 8. Adapted from (Märcher-Rorsted et al., 2021). To a solution of 8.81 g (31.77 mmol) N-trifluoroacetyl-2,5-dimethoxyphenethylamine (6) in 50 mL DCM anhydr. (note: this corresponds to a 2.5 times higher volume than given in the lit. reference and was required to keep 6 in solution) at −78° C. under nitrogen was added dropwise (4 minutes) 8.72 mL (79.45 mmol; 2.5 eq) TiCl₄. After stirring for 5 minutes, 8.04 mL (95.35 mmol; 3 eq) dichloromethyl methyl ether were added within 5 minutes; occasionally the flask was cautiously stirred manually due to the difficulty of magnetic stirring. After completion of addition stirring was continued whereby the mixture was allowed gradually to reach approx. −40° C. within 45 minutes and then the mixture was quenched by pouring it onto 100 g crushed ice. The mixture was diluted with 100 mL DCM and stirred until all solids went into solution (about 20 minutes). The two clear layers were separated, and the aq. layer (without adjusting pH) was further extracted with DCM (2×50 mL). The combined org. layers were washed with water (3×100 mL), dried over Na₂SO₄, and directly filtered and eluted through a small silica gel pad (d: 5.5 cm, h: 0.5 cm) by using additional DCM (0.5 L; end of elution checked by TLC). The eluate was concentrated in vacuo to get 8.87 g (91.4%) product 8 as a beige solid. ¹H-NMR (CDCl₃): 2.99 (t, ArCH₂), 3.63 (q, CH₂NH), 3.88 (s, MeO), 3.91 (s, MeO), 6.82 (s, 1 arom. H), 6.91 (bs, NH), 7.35 (s, 1 arom. H), 10.41 (s, CHO).

N-Trifluoroacetyl-2,5-dimethoxy-4-formylamphetamine, 9. In a similar way as described for the preparation of compound 8, to 7.40 g (25.4 mmol) N-trifluoroacetyl-2,5-dimethoxyamphetamine (7) in 140 mL DCM anhydr. (note: during cooling to −78° C., at −20° C., a suspension was formed and thus the total volume of DCM was further increased to the indicated volume in order to maintain the slurry stirrable) at −30° C. under nitrogen was added dropwise the first 0.5 mL of totally 7.0 mL (63.5 mmol; 2.5 eq) TiCl₄, which caused ease of stirring. While continuously cooling down in a cooling bath at −78° C., the remaining amount of TiCl₄ was added over a period of 10 minutes. After stirring for additional 5 minutes, to the solution were added 6.74 mL (76.2 mmol; 3 eq) dichloromethyl methyl ether over a period of 3 minutes. Next, the mixture was allowed gradually to reach approx. −35° C. within 60 minutes, and then the mixture was quenched by pouring it onto 100 g crushed ice. The mixture was diluted with 150 mL DCM and stirred until all solids went into solution (about 20 minutes). The two clear layers were separated, and the aq. layer (without adjusting pH) was further extracted with DCM (2×150 mL). The combined org. layers were washed with water (3×100 mL), dried over Na₂SO₄, and concentrated in vacuo (note: the crude product's purification could be proceeded directly as described under the preparation of compound 8, but due to time plans it was first concentrated in vacuo). The residual brownish solid was re-dissolved in DCM and eluted through silica gel (d: 3 cm, h: 8 cm) by using DCM (this needed a large 10 L □recycled□; end of elution checked by TLC). The eluate was concentrated in vacuo to get 7.89 g (97.3%) product 9 as a white solid. ¹H-NMR (CDCl₃): 1.33 (d, MeCH), 2.94 (m, ArCH₂), 3.89 (s, MeO), 3.91 (s, MeO), 4.22 (m, CHNH), 6.81 (s, 1 arom. H), 7.11 (bs, NH), 7.36 (s, 1 arom. H), 10.43 (s, CHO).

N-Trifluoroacetyl-4-acetyl-2,5-dimethoxyphenethylamine, 14. To a solution of 600 mg (2.16 mmol) N-trifluoroacetyl-2,5-dimethoxyphenethylamine (6) in 6 mL DCM anhydr. at −78° C. under nitrogen was added dropwise (3 minutes) 0.593 mL (5.41 mmol; 2.5 eq) TiCl₄. After stirring for 15 minutes, 7.56 mL (7.56 mmol; 3 eq) 1M acetyl chloride in DCM were added within 5 minutes. After completion of addition the mixture was allowed gradually to reach ambient temperature and then further stirred for 3 hours. Next, the mixture was quenched by pouring it onto 20 g crushed ice. The mixture was diluted with 20 mL DCM and stirred until discoloration and dissolution was complete. The two clear layers were separated, and the aq. layer was further extracted with DCM (2×20 mL). The combined org. layers were washed with water (1×50 mL), dried over Na₂SO₄ and concentrated in vacuo. The residual crude product was purified by silica gel chromatography (d: 4 cm, h: 15 cm) using hexane/ethyl acetate 2:1 as eluent to get 720 mg (96.4%) product 14 as a slight yellowish solid. ¹H-NMR (CDCl₃): 3.64 (s, MeCO), 2.97 (t, ArCH₂), 3.61 (q, CH₂NH), 3.86 (s, MeO), 3.89 (s, MeO), 6.79 (s, 1 arom. H), 6.95 (bs, NH), 7.37 (s, 1 arom. H).

3.) Wittig Reactions: Preparation of Alkene Intermediates 10a-f, 11a-f, 15, 17, 19-20, 23, 25a, and 25b

N-Trifluoroacetyl-2,5-dimethoxy-4-vinylphenethylamine, 10a. To an ice-cooled suspension of 2.16 g (2.3 eq) methyltriphenylphosphonium bromide in 25 mL THF anhydr. was added 2.36 mL (2.2 eq) BuLi 2.5M for 2 minutes (immediate coloration towards a deep orange). After stirring for 10 minutes at 0° C., a solution of 0.80 g (2.63 mmol) aldehyde 8 in 12 mL THF anhydr. was added over 3 minutes whereby the reaction mixture did not discolor (and thus, some of the Wittig reagent still was intact, e.g., the excess, and was not “over”-consumed). After stirring for 3 hours at 0° C., 2 mL acetone were added and the mixture was concentrated in vacuo. The residue was partitioned between ethyl acetate and water (100 mL, each), and the organic layer was dried over Na₂SO₄ and concentrated in vacuo. The crude product was redissolved in a minimal amount of DCM and coated on a silica gel column (d: 4 cm, h: 13 cm) preconditioned with hexane/ethyl acetate 9:1 and eluted with the same solvent system from 9:1 to 4:1 to get 710 mg (89.1%) product 10a as a white solid. ¹H-NMR (CDCl₃): 2.92 (t, ArCH₂), 3.59 (q, CH₂NH), 3.82 (s, MeO), 3.87 (s, MeO), 5.30 (dxd, 1H, H₂C═), 5.74 (dxd, 1H, H₂C═), 6.69 (s, 1 arom. H), superimposed: 7.03 (s, 1 arom. H), 7.04 (dxd, 1H, ArCH═) and ˜7.05 (bs, NH).

N-Trifluoroacetyl-2,5-dimethoxy-4-vinylamphetamine, 11a. It was exactly followed the procedure described for the preparation of compound 10a, by using 2.16 g (2.3 eq) methyltriphenylphosphonium bromide as the Wittig salt and 0.839 g (2.63 mmol) aldehyde 9 (reaction time: 3 hours under ice-cooling, then overnight at ambient temperature); there were obtained 690 mg (82.7%) product 11a as a white solid. ¹H-NMR (CDCl₃): 1.29 (d, MeCH), 2.87 (m, ArCH₂), 3.83 (s, MeO), 3.88 (s, MeO), 3.82 (s, MeO), 3.87 (s, MeO), 4.17 (m, CHNH), 5.32 (dxd, 1H, H₂C═), 5.74 (dxd, 1H, H₂C═), 6.67 (s, 1 arom. H), superimposed: 7.03 (s, 1 arom. H) and 7.04 (dxd, 1H, ArCH═), 7.43 (bs, NH).

N-Trifluoroacetyl-2,5-dimethoxy-4-(2,2-dimethylvinyl)phenethylamine, 10b. It was exactly followed the procedure described for the preparation of compound 10a, by using isopropyltriphenylphosphonium iodide as the Wittig salt (reaction time: 6 hours under ice-cooling, then overnight at ambient temperature). The crude product was purified by silica gel chromatography (hexane/EtOAc 9:1). From 0.80 g (2.63 mmol) aldehyde 8 there were obtained 510 mg (58.5%) product 10b as a white solid. ¹H-NMR (CDCl₃) revelated an approx. 90:10 mixture of cis/trans amide mixture (isomers not assigned; only major product peak's shifts given): 1.85 (d, 3H, MeC═), 1.95 (d, 3H, MeC═), 2.92 (t, ArCH₂), 3.59 (q, CH₂NH), 3.80 (s, MeO), 3.83 (s, MeO), 6.30 (s, ArCH═), 6.67 (s, 1 arom. H), 6.78 (s, 1 arom. H), 7.14 (bs, NH).

N-Trifluoroacetyl-2,5-dimethoxy-4-(2,2-dimethylvinyl)amphetamine, 11b. It was followed exactly the procedure described for the preparation of compound 10a, by using isopropyltriphenylphosphonium iodide as the Wittig salt (reaction time: 4 hours under ice-cooling, then overnight at ambient temperature). The crude product was purified by silica gel chromatography (hexane/EtOAc 9:1 to 5:1). From 0.839 g (2.63 mmol) aldehyde 9 there were obtained 670 mg (73.8%) product 11b as a white solid. ¹H-NMR (CDCl₃): 1.30 (d, MeCH), 1.84 (d, 3H, MeC═), 1.95 (d, 3H, MeC═), 2.87 (m, ArCH₂), 3.81 (s, MeO), 3.84 (s, MeO), 4.17 (m, CHNH), 6.30 (s, ArCH═), 6.65 (s, 1 arom. H), 6.78 (s, 1 arom. H), 7.49 (bs, NH).

Z- and E-isomer of N-trifluoroacetyl-2,5-dimethoxy-4-(prop-1-enyl)-phenethylamine, 10c and 10d. It was followed exactly the procedure described for the preparation of compound 10a, by using ethyltriphenylphosphonium bromide as the Wittig salt (reaction time: 3.5 hours under ice-cooling). The crude product was purified by silica gel chromatography (hexane/EtOAc 9:1 to 4:1). From 0.80 g (2.63 mmol) aldehyde 8 there were obtained 680 mg (81.5%) products 10c and 10d as a white solid in a 41:59 mixture of Z:E isomers (determined by NMR). ¹H-NMR (CDCl₃): For assignment of E/Z isomerism it was followed the comprehensive work of Byrne and Gilheany (Byrne & Gilheany, 2012). Z-isomer 10c: 1.87 (dxd, MeCH═), 2.92 (m, (superimposed t), ArCH₂), 3.59 (“p”(superimposed q), CH₂NH), 3.81 (s, MeO), 3.85 (s, MeO), 5.88 (dxm, 1H, MeHC═), 6.54 (dxm, 1H, ArHC═), 6.70 (s, 1 arom. H), 6.96 (s, 1 arom. H), 7.11 (bs, NH). E-isomer 10d: 1.93 (dxd, MeCH═), 2.92 (m, (superimposed t), ArCH₂), 3.59 (“p” (superimposed q), CH₂NH), 3.83 (s, MeO), 3.86 (s, MeO), 6.25 (dxq, 1H, MeHC═), 6.71 (dxm, 1H, ArHC═), 6.67 (s, 1 arom. H), 6.86 (s, 1 arom. H), 7.11 (bs, NH).

Z- and E-isomer of N-trifluoroacetyl-2,5-dimethoxy-4-(prop-1-enyl)amphetamine, 11c and 11d. It was followed exactly the procedure described for the preparation of compound 10a, by using ethyltriphenylphosphonium bromide as the Wittig salt (reaction time: 4 hours under ice-cooling, then overnight at ambient temperature). The crude product was purified by silica gel chromatography (hexane/EtOAc 9:1 to 5:1). From 0.839 g (2.63 mmol) aldehyde 9 there were obtained 610 mg (70.0%) products 11c and 11d as a white solid in a 38:62 mixture of Z:E isomers (determined by NMR). ¹H-NMR (CDCl₃): For assignment of E/Z isomerism it was followed the comprehensive work of Byrne and Gilheany (Byrne & Gilheany, 2012 10960). ¹H-NMR (CDCl₃), Z-isomer 10c: 1.29 (d (superimposed), MeCHN), 1.86 (dxd, MeCH═), 2.86 (m (superimposed), ArCH₂), 3.81 (s, MeO), 3.85 (s, MeO), 4.14 (m (superimposed), CHNH), 5.88 (dxm, 1H, MeHC═), 6.53 (dxm, 1H, ArHC═), 6.68 (s, 1 arom. H), 6.84 (s, 1 arom. H), 7.46 (bs, NH). E-isomer 10d: 1.30 (d (superimposed), MeCHN), 1.93 (dxd, MeCH═), 2.86 (m (superimposed), ArCH₂), 3.82 (s, MeO), 3.88 (s, MeO), 4.14 (m (superimposed), CHNH), 6.25 (dxq, 1H, MeHC═), 6.71 (dxm, 1H, ArHC═), 6.65 (s, 1 arom. H), 6.96 (s, 1 arom. H), 7.11 (bs, NH).

Z- and E-isomer of N-trifluoroacetyl-4-(buta-1,3-dienyl)-2,5-dimethoxy-phenethylamine, 10e and 10f. It was followed exactly the procedure described for the preparation of compound 10a, by using allyltriphenylphosphonium bromide as the Wittig salt (reaction time: 3.5 hours under ice-cooling). The crude product was purified by silica gel chromatography (hexane/EtOAc 9:1 to 4:1). From 0.80 g (2.63 mmol) aldehyde 8 there were obtained 560 mg (64.6%) products 10e and 10f as a white solid in a 62:38 mixture of double bond isomers (determined by NMR). ¹H-NMR (CDCl₃): For assignment of E/Z isomerism it could not be followed the comprehensive work of Byrne and Gilheany (Byrne & Gilheany, 2012 10960) due to a too complex superimposition of the two isomers. 2.93 (t (superimposed), ArCH₂), 3.59 (“q” (superimposed q), CH₂NH), 3.81 (s, MeO, major signal), 3.84 (s, MeO, minor signal), 3.85 (s, MeO, mayor signal), 3.89 (s, MeO, minor signal), 5.21 (dxm, 1H, H₂C═), 5.38 (dxm, 1H, H₂C═), 6.27-7.03 ((complex superimposition) m, 5H: 3× vinylic H plus 2× arom. H), 7.08 (bs, NH).

Z- and E-isomer of N-trifluoroacetyl-4-(buta-1,3-dienyl)-2,5-dimethoxy-amphetamine, 11e and 11f. It was followed exactly the procedure described for the preparation of compound 10a, by using allyltriphenylphosphonium bromide as the Wittig salt (reaction time: 4 hours under ice-cooling, then overnight at ambient temperature). The crude product was purified by silica gel chromatography (hexane/EtOAc 9:1 to 5:1). From 0.839 g (2.63 mmol) aldehyde 9 there were obtained 588 mg (65.1%) products 11e and 11f as a white solid in an approx. 60:40 ratio of double bond isomers (determined by NMR). For assignment of E/Z isomerism it could not be followed the comprehensive work of Byrne and Gilheany (Byrne & Gilheany, 2012) due to a too complex superimposition of the two isomers. ¹H-NMR (CDCl₃): 1.29 (two superimposed d, MeCH), 2.87 (m, ArCH₂), 3.81 (s, MeO, major signal), 3.83 (s, MeO, minor signal), 3.85 (s, MeO, major signal), 3.89 (s, MeO, minor signal), 4.17 (m, CHNH), 5.22 (m, 1H, H₂C═), 5.37 (m, 1H, H₂C═), 6.26-7.05 ((complex superimposition) m, 5H: 3× vinylic H plus 2× arom. H), 7.42 (bs, NH).

N-Trifluoroacetyl-2,5-dimethoxy-4-(1-methylvinyl)phenethylamine, 15. It was exactly followed the procedure described for the preparation of compound 10a, by using methyltriphenylphosphonium bromide as the Wittig salt (reaction time: 4 hours under ice-cooling, then overnight at ambient temperature) and N-trifluoroacetyl-4-acetyl-2,5-dimethoxyphenethylamine (14) as carbonyl compound. The crude product was purified by silica gel chromatography (hexane/EtOAc 4:1 to 2:1). From 0.70 g (2.19 mmol) aldehyde 14 there were obtained 495 mg (71.2%) product 15 as a white solid. ¹H-NMR (CDCl₃): 2.14 (m, MeC═), 2.92 (t, ArCH₂), 3.59 (q, CH₂NH), 3.80 (s, MeO), 3.85 (s, MeO), 5.09 (m, 1H, H₂C═), 5.18 (m, 1H, H₂C═), 6.68 (s, 1 arom. H), 6.77 (s, 1 arom. H), 7.13 (bs, NH).

N-Trifluoroacetyl-4-(2,2-difluorovinyl)-2,5-dimethoxyphenethylamine, 17. 1.) Preparation of the Wittig reagent (triphenylphosphonio)difluoroacetate (Zheng et al., 2013): In a 250 mL flask, 31.5 g (120 mmol) triphenylphosphine and 25.6 g (120 mmol) potassium bromodifluoroacetate were placed and mixed with DMF anhydr. Under nitrogen at ambient temperature. The solution was stirred for 23 hours whereby a suspension was formed progressively (product plus KBr). The suspension was filtered off, and the solid was washed with DMF (2×10 mL), H₂O (2×10 mL) and Et₂O (3×20 mL), and finally dried in vacuo at 40° C. for 20 hours to get 34.1 g (79.8%) (triphenylphosphonio)difluoroacetate as a white powder. ¹H-NMR (CD₃OD) 7.75-7.81 (m, 6H), 7.86-7.98 (m, 9H). ¹⁹F-NMR (CD₃OD) −96.00 (d, 2F, J=95.9 Hz). 2.) Wittig-type reaction to access compound 17. A mixture of 0.80 g (2.62 mmol) N-trifluoroacetyl-2,5-dimethoxy-4-formylphenethylamine (8) and 1.87 g (5.24 mmol) (triphenylphosphonio)difluoroacetate in 10 mL DMF anhydr. was heated to 50° C. It was essential not to overheat in order not to allow decomposition of the reagent in presence of a protic amide-H. After 2.5 h another 0.9eq of (triphenylphosphonio)difluoroacetate was added and heating was continued for another 1 h to complete reaction. Next, the mixture was cooled to ambient temperature and diluted with 50 mL dichloromethane (DCM), washed with water (80 mL), and the aq. layer was further extracted with DCM (1×50 mL). The combined org. layers were washed with water (3×50 mL), dried over Na₂SO₄, and concentrated in vacuo (60° C. to remove most of residual DMF) to get 2.9 g of a brown oil that was purified by silica gel chromatography (hexane/EtOAc 9:1 to 4:1). Yield: 0.476 g (53.5%) N-trifluoroacetyl-4-(2,2-difluorovinyl)-2,5-dimethoxy-phenethylamine (17) as a white solid. ¹H-NMR (CDCl₃): 2.92 (t, ArCH₂), 3.59 (q, CH₂NH), 3.81 (s, MeO), 3.85 (s, MeO), 5.67 (dd, ³J(H, F)=25.5 Hz and 5.7 Hz, F₂CCH), 6.69 (s, 1 arom. H), 7.05 (s, 1 arom. H), 7.05 (bs, NH). ¹⁹F-NMR (CDCl₃): −76.13 and −76.16 (3F, cis/trans amide), −83.19 (2F).

N-Trifluoroacetyl-4-(2,2-difluorovinyl)-2,5-dimethoxyamphetamine, 23. A mixture of 0.80 g (2.50 mmol) N-trifluoroacetyl-2,5-dimethoxy-4-formylamphetamine (9) and 1.87 g (5.24 mmol) (triphenylphosphonio)difluoroacetate (see under description of compound 17 for its preparation) in 10 mL DMF anhydr. was heated to 50° C. It was essential not to overheat in order not to allow decomposition of the reagent in presence of a protic amide-H. After 2.5 h another 0.4eq of (triphenylphosphonio)difluoroacetate was added and heating was continued for another 1 h to complete reaction. Next, the mixture was cooled to ambient temperature and diluted with 50 mL dichloromethane (DCM), washed with water (80 mL), and the aq. layer was further extracted with DCM (1×50 mL). The combined org. layers were washed with water (3×50 mL), dried over Na₂SO₄, and concentrated in vacuo (60° C. to remove most of residual DMF) to get 2.4 g of a brownish solid that was purified by silica gel chromatography (hexane/EtOAc 9:1 to 4:1). Yield: 0.519 g (58.7%) N-trifluoroacetyl-4-(2,2-difluorovinyl)-2,5-dimethoxyamphetamine (23) as a white solid. ¹H-NMR (CDCl₃) revelated an approx. 90:10 mixture of cis/trans amide mixture (isomers not assigned; only major product peak's shifts given): 1.30 (d, MeCH), 2.86 (m, ArCH₂), 3.81 (s, MeO), 3.85 (s, MeO), 4.15 (m, CHNH), 5.67 (dd, ³J(H, F)=24.3 Hz and 7.2 Hz, F₂CCH), 6.67 (s, 1 arom. H), 7.04 (s, 1 arom. H), 7.37 (bs, NH). ¹⁹F-NMR (CDCl₃): −76.31 (3F), −83.14 (2F).

E- and Z-isomers of N-trifluoroacetyl-2,5-dimethoxy-4-(2-fluorovinyl)-phenethylamine, 19 and 20. To an ice-cooled mixture of 0.80 g (2.62 mmol) N-trifluoroacetyl-2,5-dimethoxy-4-formylphenethylamine (8) and 1.01 g (2.62 mmol) (fluoromethyl)-triphenylphosphonium tetrafluoroborate (commercially available) in 15 mL THF anhydr. were added 2.75 mL (2.75 mmol) lithium hexamethyldisilazane (LiHMDS) 1M in THF over the course of 10 min. The ice bath was removed, and the mixture was stirred for 3.5 h, again cooled with an ice-bath and quenched with 10 g crashed ice. After vigorously stirring for 5 min the THF was removed in vacuo and the residue was partitioned between water (20 mL) and ethyl acetate (40 mL). The org. layer was washed with water (2×20 mL), dried over Na₂SO₄, and concentrated in vacuo. The residue was purified by silica gel chromatography (hexane/EtOAc 95:5 to 4:1). There were obtained 267 mg (31.7%) E-N-trifluoroacetyl-4-(2-fluorovinyl)-2,5-dimethoxyphenethylamine (19; white solid) as the polar compound and 177 mg (21.0%) Z—N-Trifluoroacetyl-4-(2-fluorovinyl)-2,5-dimethoxyphenethyl-amine (20; white solid) as the non-polar compound. ¹H-NMR (CDCl₃): for assignment of E/Z isomerism it was followed the work of Schlosser and Zimmermann (Schlosser & Zimmermann, 1969). E-isomer (19): 2.92 (m, ArCH₂), 3.58 (q, CH₂NH), 3.83 (s, MeO), 3.84 (s, MeO), 6.47 (dd, ³J(H, F)=22.2 Hz, ³J(H, H)=11.2 Hz, ArHC═), 6.69 (s, 1 arom. H), 6.73 (s, 1 arom. H), 7.03 (bs, NH), 7.44 (dd, ²J(H, F)=85.8 Hz, ³J(H, H)=11.1 Hz, FHC═). ¹⁹F-NMR (CDCl₃): −76.15 (3F), −124.37 (1F). Z-isomer (20): 2.92 (m, ArCH₂), 3.58 (q, CH₂NH), 3.81 (s, MeO), 3.84 (s, MeO), 6.06 (dd, ³J(H, F)=46.5 Hz, ³J(H, H)=5.6 Hz, ArHC═), 6.69 (s, 1 arom. H), 6.70 (dd, ²J(H, F)=84.0 Hz, ³J(H, H)=5.6 Hz, FHC═), 7.12 (bs, NH), 7.41 (s, 1 arom. H). ¹⁹F-NMR (CDCl₃): −76.16 (3F), −123.44 (1F).

E/Z—N-Trifluoroacetyl-2,5-dimethoxy-4-(2-fluorovinyl)amphetamine, 25a and 25b. It was exactly followed the procedure described under the preparation of compounds 19 and 20. From 0.80 g (2.50 mmol) N-trifluoroacetyl-2,5-dimethoxy-4-formylamphetamine (9) there were obtained 498 mg (59.4%) E/Z—N-trifluoroacetyl-4-(2-fluorovinyl)-2,5-dimethoxyphenethylamine (according to HPLC, LCMS and NMR analysis this was a 55:45 mixture of 25a and 25b) as a white solid (note: purification by silica gel chromatography (hexane/EtOAc) allowed no separation of the configurational E/Z-isomers). ¹H-NMR (CDCl₃): for assignment of E/Z isomerism it was followed the work of Schlosser and Zimmermann (Schlosser & Zimmermann, 1969) as well as data from separated E and Z isomers of the phenethylamines counterparts 19 and 20 was used for interpretation. Where no definite allocation could be done for E and Z isomers the values are given together. 1.28/1.30 (d, MeCH), 2.87 (m (superimposed), ArCH₂), 3.82/3.85 (s, MeO), 3.84/3.86 (s, MeO), 4.16 (m (superimposed), CHNH), 6.07 (dd, ³J(H, F)=46.2 Hz, ³J(H, H)=5.4 Hz, Z-isomer ArHC═), 6.47 (dd, ³J(H, F)=22.2 Hz, ³J(H, H)=11.1 Hz, E-isomer ArHC═), 6.68 (s, 1 arom. H E-isomer and 1 arom. H Z-isomer), 6.70 (dd, ²J(H, F)=83.7 Hz, ³J(H, H)=5.4 Hz, Z-isomer FHC═), 6.73 (s, 1 arom. H E-isomer), 7.33/7.44 (bs, NH), 7.41 (s, 1 arom. H Z-isomer), 7.44 (dd, ²J(H, F)=85.8 Hz, ³J(H, H)=11.1 Hz, E-isomer FHC═). ¹⁹F-NMR (CDCl₃): −76.33 (3F), −123.34/−124.33 (1F).

4.) N-Trifluoroacetate Hydrolysis and Hydrochloride Salt Formation: Preparation of Final Compounds 12a-f, 13a-f, 16, 18, 21-22, 24, 26a and 26b

2,5-Dimethoxy-4-vinylphenethylamine hydrochloride (2C-V), 12a. To a solution of 710 mg (2.34 mmol) 10a in 50 mL MeOH were added 20 mL aq. NaOH 2M under nitrogen. After stirring for 2 hours, the MeOH was removed by using a rotary evaporator (40° C.), and the residue was diluted with 50 mL MTBE. The layers were separated, and the org. layer was washed with 2×20 mL water, dried over Na₂SO₄ and concentrated in vacuo to get the free base of 12a (note: purging of the rotary evaporator was performed with nitrogen to prevent any possible carbamate formation). This was converted to its hydrochloride salt by applying the method described under General method for the hydrochloride salt formations. Yield: 350 mg (61.3%) product 12a as an off-white solid. ¹H-NMR (D₂O): 3.01 (t, ArCH₂), 3.28 (t, CH₂NH₃_{⁺}), 3.87 (s, MeO), 3.90 (s, MeO), 5.41 (dxd, 1H, H₂C═), 5.89 (dxd, 1H, H₂C═), 7.00 (s, 1 arom. H), 7.03 (m (superimposed), 1H, ArCH═), 7.23 (s, 1 arom. H).

2,5-Dimethoxy-4-vinylamphetamine hydrochloride (DOV), 13a. It was followed the procedure described under the preparation of compound 12a (hydrolysis and hydrochloride salt formation). From 690 mg 11a there were obtained 391 mg (69.9%) product 13a as an off-white solid. ¹H-NMR (D₂O): 1.32 (d, MeCH), 2.94 (d, ArCH₂), 3.65 (m, CHNH₃_{⁺}), 3.85 (s, MeO), 3.87 (s, MeO), 5.39 (d, 1H, H₂C═), 5.87 (d, 1H, H₂C═), 6.94 (s, 1 arom. H), 7.01 (m (superimposed), 1H, ArCH═), 7.20 (s, 1 arom. H).

2,5-Dimethoxy-4-(2,2-dimethylvinyl)phenethylamine hydrochloride (2C-DMV), 12b. It was followed the procedure described under the preparation of compound 12a (hydrolysis and hydrochloride salt formation). From 510 mg 10b there were obtained 383 mg (91.5%) product 12b as an off-white solid. ¹H-NMR (D₂O): 1.77 (d, 3H, MeC═), 1.94 (d, 3H, MeC═), 3.01 (t, ArCH₂), 3.27 (t, CH₂NH₃_{⁺}), 3.82 (s, MeO), 3.85 (s, MeO), 6.23 (s, ArCH═), 6.91 (s, 1 arom. H), 6.96 (s, 1 arom. H).

2,5-Dimethoxy-4-(2,2-dimethylvinyl)amphetamine hydrochloride (DODMV), 13b. It was followed the procedure described under the preparation of compound 12a (hydrolysis and hydrochloride salt formation). From 670 mg 11 b there were obtained 390 mg (70.5%) product 13b as an off-white solid. ¹H-NMR (D₂O): 1.32 (d, MeCH), 1.77 (s, 3H, MeC═), 1.93 (s, 3H, MeC═), 2.95 (d, ArCH₂), 3.68 (m, CHNH₃_{⁺}), 3.81 (s, MeO), 3.84 (s, MeO), 6.24 (s, ArCH═), 6.91 (s, 1 arom. H), 6.93 (s, 1 arom. H).

Z- and E-isomers of 2,5-dimethoxy-4-(prop-1-enyl)phenethylamine hydrochloride (Z-2C-MV and E-2C-MV), 12c and 12d. It was followed the procedure described under the preparation of compound 12a (hydrolysis and hydrochloride salt formation). From 680 mg of 10c and 10d (41:59 mixture of Z:E isomers) there were obtained 465 mg (84.3%) of a 39:61 mixture (determined by NMR) of product 12c and 12d as an off-white yellowish solid. ¹H-NMR (D₂O): For assignment of E/Z isomerism it was followed the comprehensive work of Byrne and Gilheany (Byrne & Gilheany, 2012). Z-isomer 12c: 1.79 (dxd, MeCH═), 3.00 (m (superimposed), ArCH₂), 3.28 (m (superimposed), CH₂NH₃_{⁺}), 3.83 (s, MeO), 3.86 (s, MeO), 5.97 (m, 1H, MeHC═), 6.47 (m (partially superimposed), 1H, ArHC═), 6.94 (s, 1 arom. H), 6.98 (s (superimposed), 1 arom. H). E-isomer 12d: 1.91 (dxd, MeCH═), 3.00 (m (superimposed), ArCH₂), 3.28 (m (superimposed), CH₂NH₃_{⁺}), 3.83 (s, MeO), 3.86 (s, MeO), 6.39 (m (partially superimposed), 1H, MeHC═), 6.67 (dxd, 1H, ArHC═), 6.98 (s (superimposed), 1 arom. H), 7.11 (s, 1 arom. H).

Z- and E-isomers of 2,5-dimethoxy-4-(prop-1-enyl)amphetamine hydrochloride (Z-DOMV and E-DOMV), 13c and 13d. It was followed the procedure described under the preparation of compound 12a (hydrolysis and hydrochloride salt formation). From 610 mg 11c and 11d (48:62 mixture of Z:E isomers) there were obtained 390 mg (78.0%) of a 35:65 mixture (determined by NMR) of product 13c and 13d as a yellowish solid. For assignment of E/Z isomerism it was followed the comprehensive work of Byrne and Gilheany (Byrne & Gilheany, 2012). ¹H-NMR (D₂O), Z-isomer 13c: 1.33 (d(superimposed), MeCHN), 1.79 (dxd, MeCH═), 2.95 (m (superimposed), ArCH₂), 3.68 (m (superimposed), CHNH₃_{⁺}), 3.83 (s, MeO), 3.85 (s, MeO), 5.88 (dxm, 1H, MeHC═), 6.48 (m (superimposed), 1H, ArHC═), 6.96 (s, 1 arom. H), 6.99 (s, 1 arom. H). E-isomer 13d: 1.31 (d (superimposed), MeCHN), 1.91 (dxd, MeCH═), 2.95 (m (superimposed), ArCH₂), 3.68 (m (superimposed), CHNH₃_{⁺}), 3.84 (s, MeO), 3.86 (s, MeO), 6.41 (m (superimposed), 1H, MeHC═), 6.68 (dxd, 1H, ArHC═), 6.93 (s, 1 arom. H), 7.14 (s, 1 arom. H).

Z- and E-isomers of 4-(buta-1,3-dienyl)-2,5-dimethoxyphenethylamine hydrochloride (Z-2C-BD and E-2C-BD), 12e and 12f. It was followed the procedure described under the preparation of compound 12a (hydrolysis and hydrochloride salt formation). From 560 mg 10e and 10f (a 62:38 mixture of double bond isomers) 205 mg (44.7%) product 12e and 12f as an off-white yellowish solid in a 62:38 mixture of double bond isomers (determined by NMR). ¹H-NMR (D₂O): For assignment of E/Z isomerism it could not be followed the comprehensive work of Byrne and Gilheany (Byrne & Gilheany, 2012) due to a too complex superimposition of the two isomers. 3.03 (m (superimposed), ArCH₂), 3.27 (m (superimposed), CH₂NH₃_{⁺}), 3.84 (s, MeO, major signal), 3.87 (s, MeO, major signal), 3.88 (s, MeO, major signal), 3.90 (s, MeO, minor signal), 5.30 (m, 1H, H₂C═), 5.48 (m, 1H, H₂C═), 6.38-7.25 ((complex superimposition) m, 5H: 3× vinylic H plus 2× arom. H)

Z- and E-isomers of 4-(buta-1,3-dienyl)-2,5-dimethoxyamphetamine hydrochloride (Z-DOBD and E-DOBD), 13e and 13f. It was followed the procedure described under the preparation of compound 12a (hydrolysis and hydrochloride salt formation). From 588 mg 11e and 11f (an approx. 60:40 ratio of double bond isomers) there were obtained 279 mg (57.5%) of product 13e and 13f as a yellowish solid in a 62:38 mixture of double bond isomers (determined by HPLC/MS). For assignment of E/Z isomerism it could not be followed the comprehensive work of Byrne and Gilheany (Byrne & Gilheany, 2012) due to a too complex superimposition of the two isomers ¹H-NMR (D₂O): 1.32 (two superimposed d, MeCH), 2.97 (t, ArCH₂), 3.67 (m (superimposed), CHNH₃_{⁺}), 3.82/3.84/3.85/3.87 (s, 2×MeO of each isomer), 5.29 (t, 1H, H₂C═), 5.46 (t, 1H, H₂C═), 6.37-7.25 ((complex superimposition) m, 5H: 3× vinylic H plus 2× arom. H).

2,5-Dimethoxy-4-(1-methylvinyl)phenethylamine hydrochloride (2C-1MV), 16. It was followed the procedure described under the preparation of compound 12a (hydrolysis and hydrochloride salt formation). From 480 mg 15 there were obtained 302 mg (77.6%) product 16 as an off-white solid. ¹H-NMR (D₂O): 2.11 (s, 3H, MeC═), 2.92 (t, ArCH₂), 3.28 (t, CH₂NH₃_{⁺}), 3.85 (s, MeO), 3.87 (s, MeO), 5.11 (t, 1H, H₂C═), 5.29 (t, 1H, H₂C═), 6.98 (s, 1 arom. H), 7.01 (s, 1 arom. H).

E-2,5-Dimethoxy-4-(2-fluorovinyl)phenethylamine hydrochloride (E-2C-FV), 21. It was followed the procedure described under the preparation of compound 12a (hydrolysis and hydrochloride salt formation). From 255 mg 19 there were obtained 170 mg (82.2%) product 21 as a yellowish solid. ¹H-NMR (D₂O): for assignment of E/Z isomerism it was followed the work of Schlosser and Zimmermann (Schlosser & Zimmermann, 1969). 3.00 (t, ArCH₂), 3.27 (t, CH₂NH₃_{⁺}), 3.85 (s, MeO), 3.86 (s, MeO), 6.56 (dd, ³J(H, F)=22.5 Hz, ³J(H, H)=11.1 Hz, ArHC═), 6.98 (s, 2 arom. H), 7.44 (dd, ²J(H, F)=85.8 Hz, ³J(H, H)=11.1 Hz, FHC═). ¹⁹F-NMR (D₂O): −125.06.

Z-2,5-Dimethoxy-4-(2-fluorovinyl)phenethylamine hydrochloride (Z-2C-FV), 22. It was followed the procedure described under the preparation of compound 12a (hydrolysis and hydrochloride salt formation). From 165 mg 20 there were obtained 95 mg (71.1%) product 22 as a yellowish solid. ¹H-NMR (D₂O): for assignment of E/Z isomerism it was followed the work of Schlosser and Zimmermann (Schlosser & Zimmermann, 1969). 3.02 (t, ArCH₂), 3.28 (t, CH₂NH₃*), 3.86 (s, MeO), 3.88 (s, MeO), 6.07 (dd, ³J(H, F)=46.5 Hz, ³J(H, H)=5.4 Hz, ArHC═), 6.86 (dd, ²J(H, F)=84.0 Hz, ³J(H, H)=5.4 Hz, FHC═), 7.02 (s, 1 arom. H), 7.43 (s, 1 arom. H). ¹⁹F-NMR (D₂O): −123.40.

E/Z-2,5-Dimethoxy-4-(2-fluorovinyl)amphetamine hydrochloride (E-DOFV and Z-DOFV), 26a and 26b. It was followed the procedure described under the preparation of compound 12a (hydrolysis and hydrochloride salt formation). From 490 mg 25a and 25b (55:45 mixture) there were obtained 335 mg (83.2%) of a 57:43 mixture of product 26a and 26b as a white solid. ¹H-NMR (D₂O): for assignment of E/Z isomerism it was followed the work of Schlosser and Zimmermann (Schlosser & Zimmermann, 1969) as well as data from separated E and Z isomers of the phenethylamine counterparts 21 and 22 was used for interpretation. Where no definite allocation could be done for E and Z isomers the values are given together. 1.33 (d, MeCH), 2.97 (m (superimposed), ArCH₂), 3.70 (m (superimposed), CHNH₃_{⁺}), 3.86/3.87/3.88 (s (superimposed), 2×MeO of each isomer), 6.07 (dd, ³J(H, F)=46.2 Hz, ³J(H, H)=5.4 Hz, Z-isomer ArHC═), 6.59 (dd, ³J(H, F)=22.8 Hz, ³J(H, H)=11.4 Hz, E-isomer ArHC═), ˜6.86 (dd (superimposed), ²J(H, F): not determinable, ³J(H, H)=5.4 Hz, Z-isomer FHC═), 6.98 (s (superimposed), 1 arom. H), 7.00 (s (superimposed), 1 arom. H), 7.02 (s (superimposed), 1 arom. H), 7.43 (s, 1 arom. H Z-isomer), 7.53 (dd (superimposed), ²J(H, F)=85.5 Hz, ³J(H, H)=11.4 Hz, E-isomer FHC═). ¹⁹F-NMR (D₂O): −123.33/−124.90.

4-(2,2-Difluorovinyl)-2,5-dimethoxyphenethylamine hydrochloride (2C-DFV), 18. Note: under classical hydrolysis conditions using aq. NaOH in MeOH a very quick decomposition occurs (MeOH/H₂O addition to the difluorovinyl group). To a warmed solution of 460 mg (1.36 mmol) 17 in 40 mL isopropanol were added 27 mL K₂CO₃ 10% aq., and the biphasic mixture was heated to 60° C. under nitrogen and vigorous stirring for 16 h. The isopropanol was removed in vacuo (50° C.; purging of the apparatus was done with nitrogen) and the residue was quickly diluted with water (30 mL), extracted with MTBE (3×30 mL), and the combined org. extracts were dried over Na₂SO₄ and concentrated in vacuo. The white residue solidified, and LCMS analysis showed that already partial decomposition of the free amine occurred (probably oligomerization by amine addition to the difluorovinyl group). Next, the residue was dissolved in 30 mL diethyl ether anhydr. containing 0.5% isopropanol and very carefully neutralized under stirring with a 0.4M HCl anhydr. solution in diethyl ether under ice-cooling and nitrogen to pH 8-9 (it was found to be much gentler to use fumaric acid for salt formation, see also under preparation of compound 24). After filtering off the suspension formed, rinsing the filter cake with diethyl ether, and drying it in vacuo there were obtained 257 mg product 22 as a yellowish solid. ¹H-NMR (D₂O): 2.90 (t, ArCH₂), 3.16 (t, CH₂NH₃_{⁺}), 3.75 (s, MeO), 3.77 (s, MeO), 5.60 (dd, ³J(H, F)=22.5 Hz and 8.4 Hz, F₂CCH), 6.90 (s, 1 arom. H), 7.05 (s, 1 arom. H). ¹⁹F-NMR (D₂O): −83.50.

4-(2,2-Difluorovinyl)-2,5-dimethoxyamphetamine hemifumarate (DODFV), 24. Note: under classical hydrolysis conditions using aq. NaOH in MeOH a very quick decomposition occurs (MeOH/H₂O addition to the difluorovinyl group). To a warmed solution of 512 mg (1.45 mmol) 23 in 40 mL isopropanol were added 27 mL K₂CO₃ 10% aq., and the biphasic mixture was heated to 75° C. under nitrogen and vigorous stirring for 4 days (note: the trifluoroacetate hydrolysis takes place at a much lower rate than observed for the 2C derivative 17 under these hydrolysis conditions). After this time reaction was complete and no decomposition products were visible in LCMS/UV. The isopropanol was removed in vacuo (50° C.; purging of the apparatus was done with nitrogen) and the residue was quickly diluted with water (30 mL), extracted with MTBE (2×30 mL), and the combined org. layers were washed with water (1×30 mL), dried over Na₂SO₄, and concentrated in vacuo (again, purged with nitrogen). LCMS analysis of the residual oil showed that a much smaller amount was decomposed than observed on the free base of compound 17 (herein, oligomerization by amine addition to the difluorovinyl group is probably slowed due to steric hindrance by presence of the alpha-methyl group). Nevertheless, a quick salt formation was performed as follows. The residue was dissolved in 30 mL diethyl ether anhydr. containing 0.1% isopropanol and very carefully neutralized with a solution of 85 mg fumaric acid (this corresponds to the theoretical max. amount of 0.5 eq) in 20 mL diethyl ether and 0.6 mL isopropanol (for easier dissolution ultrasonic was applied to dissolve fumaric acid) under ice-cooling, stirring and nitrogen to pH 8-9 (note: a slight basic pH allows prevention of over-titration and thus formation of mixed hemifumarate/fumarate salts). The gel-like suspension was filtered off under a very slight nitrogen stream and rinsing the filter cake with additional diethyl ether continuously eased solidification and filtration, whereby the filter cake was never sucked to complete dryness. The wet solids, still on the filter frit, were dried in a vacuum oven (30° C.) to get 285 mg (62.3%) 24 as a white solid. LCMS/UV revealed that under these conditions no decomposition occurred, and the product was pure, whereas ¹H-NMR (D₂O) analysis showed a perfect 1:0.50 ratio of amine to fumaric acid (hemifumarate salt). 1.27 (d, MeCH), 2.91 (m, ArCH₂), 3.57 (m, CHNH₃_{⁺}), 3.83 (s, 2 MeO), 5.70 (dd, ³J(H, F)=25.2 Hz and 6.6 Hz, F₂CCH), 6.67 (s, 1H, fumaric acid [as hemifumarate anion]), 6.87 (s, 1 arom. H), 7.07 (s, 1 arom. H). ¹⁹F-NMR (D₂O): −86.47.

### Examples—Preparation of the 4-alkynyl-2,5-dimethoxyphenethylamine Derivatives 29, 31 and 37

N-Trifluoroacetyl-2,5-dimethoxy-4-ethynylphenethylamine, 28. To a mixture of 1.0 g (2.48 mmol) N-trifluoroacetyl-2,5-dimethoxy-4-iodophenethylamine (27) (Johnson et al., 1990; Nichols et al., 1994) (caution: 27 must not be contaminated by iodine monochloride from the previous reaction), 20 mg (0.1 mmol) CuI and 35 mg (0.05 mmol) PdCl₂(PPh₃)₂ in 5 mL THF anhydr. under nitrogen was added 1.37 mL (9.9 mmol) triethylamine in one portion. After stirring for 1 min, 0.51 g (5.2 mmol) ethynyltrimethylsilane in 1 mL THF anhydr. was added during 5 min (slow addition prevents the formation of diynes). After stirring for 1 h, the conversion was complete, and the solvent was evaporated. The residue was taken up in ethyl acetate. Filtration through Celite and evaporation of the filtrate afforded a viscous oil which slowly crystallized to a beige solid. Yield: 0.90 g (97%) of the trimethylsilylethynyl intermediate. ¹H-NMR (CDCl₃): 0.30 (s, Me₃Si), 2.90 (t, ArCH₂), 3.55 (q, CH₂NH), 3.82 (s, MeO), 3.84 (s, MeO), 6.63 (s, 1 arom. H), 6.88 (bs, NH), 6.94 (s, 1 arom. H). Next, a solution of 0.87 g (2.33 mmol) trimethylsilylethynyl intermediate in 25 mL THF was treated with 1M Bu4NF in THF (11.0 mL) and stirred for 2 h. The mixture was quenched with sat. aq. NH4Cl soln. (70 mL) and extracted with AcOEt (3×40 mL), the combined extracts were washed with brine, dried (Na₂SO₄), and evaporated, and the obtained viscous brownish oil further purified by silica gel chromatography (hexane/EtOAc 2:1 to 1:1). Yield: 0.64 g (91%) 28 as a beige solid. ¹H-NMR (CDCl₃): 2.93 (t, ArCH₂), 3.34 (s, HCC), 3.59 (q, CH₂NH), 3.82 (s, MeO), 3.87 (s, MeO), 6.67 (s, 1 arom. H), 6.90 (bs, NH), 6.99 (s, 1 arom. H).

2,5-Dimethoxy-4-ethynylphenethylamine hydrochloride (2C-YN), 29. A soln. of 0.63 g (2.09 mmol) in 120 mL MeOH was treated with 35 mL 5M NaOH aq. and stirred for 4 h. The mixture was extracted with Et₂O (150 mL), the aq. phase further extracted with Et₂O (3×50 mL), and the combined org. layers washed with H2O (2×50 mL), dried (Na₂SO₄), and evaporated. The residual oil (reacts with CO₂ very quickly; should be stored under an inert gas if used as free base) was dissolved in Et₂O anhydr. (10 mL) and the solution neutralized with anhydr. 1M HCl in Et₂O. The crystals were filtered off, washed with Et₂O, and dried. Yield: 0.44 g (88%) 29 as off-white crystals. ¹H-NMR (D₂O): 2.84 (t, ArCH₂), 3.10 (t, CH₂NH₃_{⁺}), 3.61 (s, HCC), 3.66 (s, MeO), 3.71 (s, MeO), 6.80 (s, 1 arom. H), 6.97 (s, 1 arom. H).

N-Trifluoroacetyl-2,5-dimethoxy-4-(prop-1-ynyl)phenethylamine, 30. It was exactly followed the procedure described under the preparation of compound 28 by using N-trifluoroacetyl-2,5-dimethoxy-4-iodophenethylamine (27) and gaseous propyne as the alkyne reagent; a balloon filled with propyne was attached to a syringe entering into the reaction mixture, and a second syringe was attached to the reaction flask entering into the gaseous void in order to allow slow bubbling of propyne directly into the liquid reaction mixture for 3 minutes. Then the second needle was removed, and the balloon remained attached until completion of reaction (overnight). After workup there were obtained 720 mg (92%) 30 as a beige-brown solid. ¹H-NMR (CDCl₃): 2.14 (s, MeCC), 2.91 (t, ArCH₂), 3.57 (q, CH₂NH), 3.82 (s, MeO), 3.86 (s, MeO), 6.67 (s, 1 arom. H), 6.94 (s, 1 arom. H), 6.97 (bs, NH).

2,5-Dimethoxy-4-(prop-1-ynyl)phenethylamine hydrochloride (2C-PYN), 31. To a solution of 715 mg (2.27 mmol) 30 in 200 mL MeOH were added 10 mL aq. 2M NaOH solution and stirring was continued for 4 h under nitrogen. The MeOH was removed by using a rotary evaporator (40° C.), and the residue was diluted with 60 mL MTBE. The layers were separated, and the org. layer was washed with 2×20 mL water, dried over Na₂SO₄ and concentrated in vacuo to get the free base of 31 (note: purging of the rotary evaporator was performed with nitrogen to prevent any possible carbamate formation). This was converted to its hydrochloride salt by applying the method described under General method for the hydrochloride salt formations. Yield: 474 mg (75%) product 31 as an off-white solid. ¹H-NMR (D₂O): 2.01 (s, MeCC), 2.89 (t, ArCH₂), 3.16 (t, CH₂NH₃_{⁺}), 3.73 (s, MeO), 3.77 (s, MeO), 6.84 (s, 1 arom. H), 6.95 (s, 1 arom. H).

2,5-Dimethoxy-4-iodobenzaldehyde, 32. It was followed the reference (Hathaway et al., 1998), with some important changes. To a solution of 25.0 g (0.15 mol) 2,5-dimethoxybenzaldehyde in 600 mL MeOH anhydr., placed in an aluminium-foil covered reaction flask (protection from light), were added subsequently 42.0 g (0.1655 mmol) elemental iodine and 28.1 g (0.1655 mmol) AgNO₃ and the reaction mixture was stirred for 17 hours under nitrogen. The precipitated solids were removed by filtration and the filter cake was rinsed with additional MeOH. The combined filtrates were concentrated in vacuo, and the residue was suspended in water (1 L) and extracted with DCM (3×1 L). The combined org. extracts were washed with water (1 L) and brine (0.7 L), dried over Na₂SO₄ and concentrated in vacuo to get 44.3 g of a pale yellow waxy solid. ¹H-NMR in DMSO-D6 (note: CDCl₃ immediately hydrolyzes any acetal) and GCMS analysis revealed this to be pure 2,5-dimethoxy-4-iodobenzaldehyde dimethyl acetal; LCMS under acidic conditions inherently hydrolyzes any acetal and misleads to the assumption of having the corresponding aldehyde. ¹H-NMR (DMSO-D6): 3.26 (s, 6H, CH(CH₃)₂), 3.66 (s, 1 MeO), 3.75 (s, MeO), 3.76 (s, MeO), 5.49 (s, CH(OMe)₂), 6.95 (s, 1 arom. H), 7.39 (s, 1 arom. H). Next, the dimethyl acetal intermediate (44.3 g) was dissolved in 900 mL MeOH using ultrasonic before 88 mL 6M hydrochloric acid aq. was added. After stirring for 1.5 hours about half of MeOH was removed in vacuo and the residual mixture was stirred under ice-cooling for 2 hours. The precipitation was filtered off and washed with cold MeOH (2×50 mL) and water (4×200 mL; suspended well to remove any residual HCl) and finally dried at 35° C. in a high vacuum to get a first crop of 32.93 g product. The above MeOH filtrates (without water washings) were further concentrated in vacuo to about ½ of its volume and stirred in an ice-bath overnight, and the precipitation was filtered off, rinsed with cold MeOH (10 mL) and water (4×20 mL) and dried at 35° C. in high vacuum to get a second crop of 2.77 g product with identical purity as the first crop. Total yield: 35.7 g (81.5%) product 32 as a pale yellow-greenish solid. ¹H-NMR (DMSO-D6—Note: in order to ensure no acetal was present anymore NMR analysis was performed in DMSO and not in CDCl₃): 3.82 (s, MeO), 3.89 (s, MeO), 7.13 (s, 1 arom. H), 7.69 (s, 1 arom. H), 10.29 (s, CHO).

1-(2,5-Dimethoxy-4-iodophenyl)-2-nitropropene, 33. According to the general method described, from 36.4 g 2,5-dimethoxy-4-iodobenzaldehyde (32), 160 mL nitroethane, 1.45 mL butylamine, 1.45 mL acetic acid and 3.3 g molecular sieves, 60 minutes at 95° C. Yield: 36.21 g (83.2%) 33 as bright orange crystals. ¹H-NMR (CDCl₃): 2.37 (d, MeC), 3.83 (s, MeO), 3.85 (s, MeO), 6.73 (s, 1 arom. H), 7.25 (s, 1 arom. H), 8.14 (t, CH═C).

2,5-Dimethoxy-4-iodoamphetamine hydrochloride (DOI), 34. According to the general method described for the alane-promoted nitro olefin reduction, from 15.0 g 33, 6.09 g LiAlH₄, 4.23 mL H₂SO₄, 130 mL plus 60 mL THF, 25.4 mL IPA and 19.4 mL NaOH 2M. Hydrochloride salt formation according to the general method described. Yield: 11.19 g (72.8%) product as a white solid. ¹H-NMR (D₂O): 1.29 (d, MeCH), 2.89 (m, ArCH₂), 3.66 (m, CHNH₃_{⁺}), 3.80 (s, MeO), 3.83 (s, MeO), 6.87 (s, 1 arom. H), 7.42 (s, 1 arom. H).

N-Trifluoroacetyl-2,5-dimethoxy-4-iodoamphetamine, 35. To a solution of 5.29 g (14.5 mmol) 34 and 4.54 mL (32.5 mmol; 2.2 eq) NEt₃ in 40 mL MeOH anhydr. was added dropwise 1.75 mL (16.3 mmol; 1.1 eq) ethyl trifluoroacetate within 2 minutes under nitrogen. After stirring for 2 hours at ambient temperature, the volatiles were removed in vacuo and the residue was redissolved in MTBE (200 mL), washed with aq. HCl 0.5M (2×80 mL) and water (2×80 mL), dried over Na₂SO₄ and concentrated in vacuo to get 5.81 g (96.0%) product 35 as a white solid. ¹H-NMR (CDCl₃): 1.29 (d, MeCH), 2.85 (m, ArCH₂), 3.84 (s, MeO), 3.85 (s, MeO), 4.17 (m, CHNH), 6.63 (s, 1 arom. H), 7.15 (bs, NH), 7.29 (s, 1 arom. H).

N-Trifluoroacetyl-2,5-dimethoxy-4-(trimethylsilylethynyl)amphetaimine, 36. To a mixture of 2.06 g (4.96 mmol) N-trifluoroacetyl-2,5-dimethoxy-4-iodoamphetamine (35), 40 mg (0.2 mmol) CuI and 70 mg (0.1 mmol) PdCl₂(PPh₃)₂ in 20 mL THF anhydr. under nitrogen was added 2.76 mL (19.8 mmol) triethylamine in one portion. After stirring for 1 min, 1.44 mL (10.4 mmol) ethynyltrimethylsilane in 2 mL THF anhydr. was added during 10 min (slow addition prevents the formation of diynes). After stirring for 1.5 h, the conversion was complete, and the solvent was evaporated. The residue was taken up in 50 mL ethyl acetate. Filtration through a 1 cm silica gel pad, rinsing with additional ethyl acetate (130 mL) and evaporation of the filtrate afforded 2.1 g of the crude trimethylsilylethynyl intermediate 36 as a brownish solid. ¹H-NMR (CDCl₃): 0.29 (s, Me₃Si), 1.27 (d, MeCH), 2.85 (m, ArCH₂), 3.84 (s, MeO), 3.85 (s, MeO), 4.15 (m, CHNH), 6.65 (s, 1 arom. H), 6.97 (s, 1 arom. H), 7.22 (bs, NH).

2,5-Dimethoxy-4-ethynylamphetamine hydrochloride (DOYN), 37. To a solution of 2.1 g (approx. 4.9 mmol) of crude trimethylsilylethynyl intermediate 36 in 250 mL MeOH were added 10 mL K₂CO₃ 2M aq. and after stirring for 30 min under nitrogen atmosphere an aq. 2M NaOH solution (40 mL) was added and stirring was continued for 2 h. The MeOH was removed by using a rotary evaporator (40° C.), and the residue was extracted with 3×60 mL MTBE. The org. layers were combined and washed with 3×30 mL water, dried over Na₂SO₄ whereby 2 g activated charcoal pellets were added prior filtering off, and concentrated in vacuo to get the free base of 37 (note: purging of the rotary evaporator was performed with nitrogen to prevent any possible carbamate formation). This was converted to its hydrochloride salt by applying the method described under General method for the hydrochloride salt formations. Yield: 880 mg (overall 69%, from 35) product 37 as an off-white solid. ¹H-NMR (D₂O): 1.25 (d, MeCH), 2.90 (d, ArCH₂), 3.62 (m, CHNH₃_{⁺}), 3.71 (s, HCC), 3.78 (s, MeO), 3.82 (s, MeO), 6.90 (s, 1 arom. H), 7.12 (s, 1 arom. H).

### Examples—Preparation of the 4-alkenyl-2,6-dimethoxyphenethylamine Derivatives 41-42, 49-50 and 51-52

### 1.) Preparation of N-TFA Protected Phenethylamine Templates 43 and 44

4-Bromo-2,6-dimethoxy-β-nitrostyrene, 39. According to the general method for the nitro olefination described, from 8.0 g 4-bromo-2,6-dimethoxybenzaldehyde (38; commercially available), 17 mL nitromethane, 0.24 mL butylamine and 0.24 mL acetic acid, 4 g molecular sieves. This reaction was so fast that when adding butylamine and acetic acid, the warmed solution immediately caused a precipitation of product formed. To ensure completion of reaction the mixture was heated for 8 minutes in an oil bath set at 90° C. (TLC check, DCM as eluent). Yield: 8.43 g (89.7%) 39 as bright yellow crystals. ¹H-NMR (CDCl₃): 3.95 (s, 2 MeO), 6.78 (s, 2 arom. H), 8.03 (d, CHNO₂), 8.46 (d, CH═CHNO₂).

1-(4-Bromo-2,6-dimethoxyphenyl)-2-nitropropene, 40. According to the general method described for the nitro olefination, from 8.0 g 4-bromo-2,6-dimethoxybenzaldehyde (38; commercially available), 17 mL nitroethane, 0.24 mL butylamine, 0.24 mL acetic acid and 4 g molecular sieves, 20 min at 95° C. Yield: 7.76 g (78.7%) 40 as bright yellow crystals. ¹H-NMR (CDCl₃): 2.10 (d, ArCH₃), 3.86 (s, 2 MeO), 6.78 (s, 2 arom. H), 7.84 (s, CH═C).

4-Bromo-2,6-dimethoxyphenethylamine hydrochloride (ψ-2C-B), 41. According to the general method described for the alane-promoted nitro olefin reduction, from 8.10 g 39, 3.97 g LiAlH₄, 2.78 mL H₂SO₄, 85 mL plus 45 mL THF, 16.5 mL IPA and 12.7 mL NaOH 2M. The obtained free base of 41 quickly crystallized to a white solid. Hydrochloride salt formation according to the general method described. Yield: 7.49 g (89.8%) product 41 as a white solid. ¹H-NMR (D₂O): 2.91 (t, ArCH₂), 3.09 (t, CH₂NH₃_{⁺}), 3.79 (s, 2 MeO), 6.90 (s, 2 arom. H).

4-Bromo-2,6-dimethoxyamphetamine hydrochloride (W-DOB), 42. According to the general method described for the alane-promoted nitro olefin reduction, from 7.70 g 40, 3.60 g LiAlH₄, 2.52 mL H₂SO₄, 80 mL plus 30 mL THF, 15.0 mL IPA and 11.5 mL NaOH 2M. Hydrochloride salt formation according to the general method described. Yield: 7.48 g (94.6%) product 42 as a white solid. ¹H-NMR (D₂O): 1.24 (d, MeCH), 2.85 (m, ArCH₂), 3.50 (m, CHNH₃_{⁺}), 3.78 (s, 2 MeO), 6.89 (s, 2 arom. H).

N-Trifluoroacetyl-4-Bromo-2,6-dimethoxyphenethylamine, 43. To a solution of 4.0 g (13.49 mmol) 41 and 4.13 mL (2.2 eq) NEt₃ in 25 mL MeOH anhydr. was added dropwise 1.60 mL (14.84 mmol; 1.1 eq) ethyl trifluoroacetate within 1 minute under nitrogen. After stirring for 2 hours at ambient temperature, the volatiles were removed in vacuo and the residue was redissolved in MTBE (80 mL), washed with aq. HCl 0.5M (2×50 mL) and water (2×50 mL), dried over Na₂SO₄ and concentrated in vacuo to get 4.20 g (87.5%) product 43 as a beige solid. ¹H-NMR (CDCl₃): 2.93 (m, ArCH₂), 3.49 (m, CH₂NH), 3.84 (s, 2 MeO), 6.75 (s, 2 arom. H), 6.90 (bs, NH).

N-Trifluoroacetyl-4-Bromo-2,6-dimethoxyamphetamine, 44. To a solution of 4.19 g (13.49 mmol) 42 and 4.13 mL (2.2 eq) NEt₃ in 25 mL MeOH anhydr. was added dropwise 1.60 mL (14.84 mmol; 1.1 eq) ethyl trifluoroacetate within 1 minute under nitrogen. After stirring for 2 hours at ambient temperature, the volatiles were removed in vacuo and the residue was redissolved in MTBE (120 mL), washed with aq. HCl 0.5M (2×50 mL) and water (2×50 mL), dried over Na₂SO₄ and concentrated in vacuo to get 3.70 g (74.1%) product 44 as a white solid. ¹H-NMR (CDCl₃): 1.30 (d, MeCH), 2.84 (m, ArCH₂), 3.85 (s, 2 MeO), 4.07 (m, CHNH), 6.74 (s, 2 arom. H), 7.19 (bs, NH).

### 2.) Introduction of the Carbonyl Group: Preparation of Templates 45 and 46

N-Trifluoroacetyl-2,6-dimethoxy-4-formylphenethylamine, 45. To a solution of 8.9 mL (20.22 mmol, 3.0 eq) BuLi 2.5M in 40 mL THF anhydr. at −78° C. under nitrogen was added dropwise (7 minutes) a solution of 2.40 g (6.74 mmol) 43 in 26 mL THF anhydr. After stirring for 5 minutes, 6.7 mL DMF anhydr. were added within 3 minutes. After completion of addition stirring was continued for 20 minutes and then the mixture was allowed to reach −10° C. Next, the mixture was quenched by a quick addition of 50 mL aq. saturated NH₄Cl solution and 50 mL of citric acid 10% (pH check: approx. 5). The mixture was extracted with EtOAc (2×50 mL), and the combined org. extracts were washed twice with water, dried over Na₂SO₄ and concentrated in vacuo (briefly at 60° C. in order to remove any residual DMF) to get 1.94 g of a beige solid. The residue was recrystallized from EtOAc and hexane (added to the hot EtOAc solution). Yield: 770 mg (37.4%) product 45 as a beige solid. The mother liquor still contained some product, among the by-product N-trifluoroacetyl-2,6-dimethoxyphenethylamine which can be separated by column chromatography. ¹H-NMR (CDCl₃): 3.06 (m, ArCH₂), 3.56 (m, CH₂NH), 3.94 (s, 2 MeO), 6.91 (bs, NH), 7.12 (s, 2 arom. H), 9.95 (s, CHO).

N-Trifluoroacetyl-2,6-dimethoxy-4-formylamphetamine, 46. To a solution of 6.63 mL (15.06 mmol, 3.0 eq) BuLi 2.5M in 30 mL THF anhydr. at −78° C. under nitrogen was added dropwise (6 minutes) a solution of 1.83 g (5.02 mmol) 44 in 20 mL THF anhydr. After stirring for 5 minutes, 5.0 mL DMF anhydr. were added within 3 minutes. After completion of addition stirring was continued for 20 minutes and then the mixture was allowed to reach −10° C. Next, the mixture was quenched by a quick addition of 50 mL aq. saturated NH₄Cl solution and 50 mL of citric acid 10% (pH check: approx. 5). The mixture was extracted with EtOAc (2×50 mL), and the combined org. extracts were washed twice with water, dried over Na₂SO₄, and concentrated in vacuo (briefly at 60° C. in order to remove any residual DMF) to get 1.94 g of a beige solid. The residue was dissolved in a minimal amount of DCM and to this solution 12 g silica gel were added and the solvent was removed by rotary evaporation at 45° C. The residue was packed onto a silica gel dry flash column (h: 4 cm, d: 5.5 cm) preconditioned with hexane/EtOAc 95:5. It was eluted with the same eluent until all the unwanted by-product (N-trifluoroacetyl-2,6-dimethoxyamphetamine) was eluted, then it was changed to hexane/EtOAc 1:1 to elute the desired product. After pooling and evaporating the corresponding fractions, there were obtained 748 mg (48.9%) product 46 as a white solid. ¹H-NMR (CDCl₃): 1.33 (d, MeCH), 2.94 (m, ArCH₂), 3.94 (s, 2 MeO), 4.14 (m, CHNH), 7.12 (s, 2 arom. H), 7.16 (bs, NH), 9.95 (s, CHO).

### 3.) Wittig Reactions: Preparation of Alkene Intermediates 47 and 48

N-Trifluoroacetyl-2,6-dimethoxy-4-vinylphenethylamine, 47. To an ice-cooled suspension of 2.05 g (2.3 eq) methyltriphenylphosphonium bromide in 25 mL THF anhydr. was added 2.24 mL (2.2 eq) BuLi 2.5M for 2 minutes. After stirring for 10 minutes at 0° C., a solution of 0.76 g (2.49 mmol) aldehyde 45 in 10 mL THF anhydr. was added over 3 minutes whereby the reaction mixture did not discolor (and thus, some of the Wittig reagent still was intact, e.g., the excess, and was not “over”-consumed). After stirring for 5 minutes the ice bath was removed and stirring was continued for 70 minutes. Next, 2 mL acetone were added and the mixture was concentrated in vacuo. The residue was partitioned between ethyl acetate and water (60 mL, each), and the organic layer was dried over Na₂SO₄ and concentrated in vacuo. The crude beige solid (1.58 g) The crude product was redissolved in a minimal amount of DCM and coated on a silica gel column (d: 4 cm, h: 20 cm) preconditioned with hexane/ethyl acetate 9:1 and eluted with the same solvent system to get 628 mg (83.2%) product 47 as a white solid. ¹H-NMR (CDCl₃) revelated an approx. 80:20 mixture of cis/trans amide mixture (isomers not assigned; only major product peak's shifts given): 2.98 (m (superimposed), ArCH₂), 3.52 (m (superimposed), CH₂NH), 3.88 (s (partially superimposed), 2 MeO), 5.29 (dxd, 1H, H₂C═), 5.78 (dxd, 1H, H₂C═), 6.64 (s, 2 arom. H), 6.70 (dxd, 1H, ArCH═), 7.10 (bs, NH).

N-Trifluoroacetyl-2,6-dimethoxy-4-vinylamphetamine, 48. It was exactly followed the procedure described for the preparation of compound 47, by using 1.98 g (2.3 eq) methyltriphenylphosphonium bromide as the Wittig salt and 0.770 g (2.41 mmol) aldehyde 46 (reaction time: 5 minutes under ice-cooling, then 70 minutes at ambient temperature); there were obtained 571 mg (74.7%) product 48 as a white solid. ¹H-NMR (CDCl₃) revelated an approx. 90:10 mixture of cis/trans amide mixture (isomers not assigned; only major product peak's shifts given): 1.30 (d (superimposed), MeCH), 2.89 (m (superimposed), ArCH₂), 3.89 (s, 2 MeO), 4.06 (m (superimposed), CHNH), 5.29 (dxd (partially superimposed), 1H, H₂C═), 5.75 (dxd, 1H, H₂C═), 6.64 (s, 2 arom. H), 6.67 (dxd (partially superimposed), 1H, ArCH═), 7.42 (bs, NH).

### 4.) Hydrolysis and Hydrochloride Salt Formation: Preparation of Final Compounds 49 and 50

2,6-Dimethoxy-4-vinylphenethylamine hydrochloride (ψ-2C-V), 49. To a solution of 620 mg (2.04 mmol) 47 in 45 mL MeOH were added 30 mL aq. NaOH 2M under nitrogen. After stirring for 70 minutes, the MeOH was removed by using a rotary evaporator (40° C.), and the residue was diluted with 30 mL water and extracted with MTBE (3×30 mL). The combined org. layers were washed with 2×20 mL water, dried over Na₂SO₄ and concentrated in vacuo to get the free base (389 mg) of 49 (note: purging of the rotary evaporator was performed with nitrogen to prevent any possible carbamate formation). This was converted to its hydrochloride salt by applying the method described under General method for the hydrochloride salt formations. Yield: 387 mg (77.9%) product 49 as a white solid. ¹H-NMR (D₂O): 2.91 (t, ArCH₂), 3.07 (t, CH₂NH₃_{⁺}), 3.79 (s, 2 MeO), 5.28 (d, 1H, H₂C═), 5.82 (d, 1H, H₂C═), 6.69 (dxd, 1H, ArCH═), 6.76 (s, 2 arom. H).

2,6-Dimethoxy-4-vinylamphetamine hydrochloride (W-DOV), 50. To a solution of 560 mg (1.77 mmol) 48 in 45 mL MeOH were added 30 mL aq. NaOH 2M under nitrogen. After stirring for 70 minutes, the MeOH was removed by using a rotary evaporator (40° C.), and the residue was diluted with 30 mL water and extracted with MTBE (3×30 mL). The combined org. layers were washed with 2×20 mL water, dried over Na₂SO₄ and concentrated in vacuo to get the free base (355 mg) of 50 (note: purging of the rotary evaporator was performed with nitrogen to prevent any possible carbamate formation). This was converted to its hydrochloride salt by applying the method described under General method for the hydrochloride salt formations. Yield: 373 mg (82.0%) product 50 as a white solid. ¹H-NMR (D₂O): 1.22 (d, MeCH), 2.87 (m, ArCH₂), 3.50 (m, CHNH₃_{⁺}), 3.79 (s, 2 MeO), 5.28 (d, 1H, H₂C═), 5.83 (d, 1H, H₂C═), 6.70 (dxd, 1H, ArCH═), 6.77 (s, 2 arom. H).

### Examples—Preparation of the 4-alkyl-2,6-dimethoxyphenethylamine Derivatives 51 and 52

2,6-Dimethoxy-4-ethylphenethylamine hydrochloride (W-2C-E), 51. The vinyl compound 49 (120 mg, 0.492 mmol) was dissolved in 10 mL absolute ethanol under warming and some ultrasonic. Next, 15 mg palladium 10% on activated charcoal was added to the solution at ambient temperature under nitrogen, and the gaseous layer was exchanged by hydrogen (balloon, by vacuumation/ventilation technique). After vigorously stirring for 45 minutes the mixture was filtered through a 0.45 μm microfilter attached to a syringe, the syringe and filter were rinsed with additional 10 mL ethanol, and the combined filtrates were concentrated in vacuo. The solid residue was further dried on high vacuum to get 118 mg (97.6%) 51 as a white solid. ¹H-NMR (D₂O): 1.15 (t, ArCH₂CH₃), 2.58 (q, ArCH₂CH₃), 2.91 (t, ArCH₂), 3.07 (t, CH₂NH₃_{⁺}), 3.79 (s, 2 MeO), 6.61 (s, 2 arom. H).

2,6-Dimethoxy-4-ethylamphetamine hydrochloride (W-DOET), 52. The vinyl compound 50 (120 mg, 0.492 mmol) was dissolved in 10 mL absolute ethanol under warming and some ultrasonic. Next, 15 mg palladium 10% on activated charcoal was added to the solution at ambient temperature under nitrogen, and the gaseous layer was exchanged by hydrogen (balloon, by vacuumation/ventilation technique). After vigorously stirring for 45 minutes the mixture was filtered through a 0.45 μm microfilter attached to a syringe, the syringe and filter were rinsed with additional 10 mL ethanol, and the combined filtrates were concentrated in vacuo. The solid residue was further dried on high vacuum to get 115 mg (95.0%) 52 as a white solid. ¹H-NMR (D₂O): 1.15 (t, ArCH₂CH₃), 1.22 (d, MeCH), 2.59 (q, ArCH₂CH₃), 2.85 (m, ArCH₂CH), 3.49 (m, CHNH₃_{⁺}), 3.78 (s, 2 MeO), 6.60 (s, 2 arom. H).

### Examples—Preparation of the 4-alkynyl-2,6-dimethoxyphenethylamine Derivatives 55 and 56

N-Trifluoroacetyl-2,6-dimethoxy-4-(trimethylsilylethynyl)phenethylamine, 53. To a mixture of 1.77 g (4.96 mmol) 43, 40 mg (0.2 mmol) CuI and 70 mg (0.1 mmol) PdCl₂(PPh₃)₂ in 20 mL THF anhydr. under nitrogen was added 1.76 mL (19.8 mmol) triethylamine in one portion. After stirring for 1 min, 1.44 mL (10.4 mmol) ethynyltrimethylsilane in 2 mL THF anhydr. was added during 5 min (generally, slow addition prevents the formation of diynes). This reaction was essentially slow and thus, over the course of 5 days, there was added another 0.5 equals of the above amounts of palladium catalyst and alkyne on day 2, 3, 4 and 5. After stirring for a total of 7 days the solvent was evaporated. The residue was redissolved in a minimal amount of DCM and pre-coated on silica gel (8 g) and then the DCM was evaporated. The coated product was plugged on a silica gel column (d: 4 cm, h: 10 cm) preconditioned with hexane/ethyl acetate 95:5 and eluted (95:5 to 9:1) to get 114 mg (6.2%) product 53 as a brownish solid. ¹H-NMR (CDCl₃; a superimposition of either rotamers and/or cis/trans isomers of the amide function was observed. LCMS: eluted as a single compound): 0.28 (m, Me₃Si), 2.97 (m, ArCH₂), 3.49 (m, CH₂NH), 3.84 (m, 2×MeO), 6.70 (s, 2 arom. H), 6.97 (bs, NH).

2,6-Dimethoxy-4-ethynylphenethylamine hydrochloride (ψ-2C-YN), 55. To a solution of 111 mg (0.297 mmol) trimethylsilylethynyl intermediate 53 in 25 mL MeOH were added 0.5 mL K₂CO₃ 2M aq. and after stirring for 75 min LCMS indicated complete TMS removal. An aq. 2M NaOH solution (2 mL) was added and stirring was continued for 17 h. The MeOH was removed by using a rotary evaporator (40° C.), and the residue was taken up with 50 mL MTBE and washed with 3×30 mL water, dried over Na₂SO₄ whereby 2 g activated charcoal pellets were added prior filtering off, and the filtrate was concentrated in vacuo to get the free base of 55 (84 mg, solidified over 3 hours; note: purging of the rotary evaporator was performed with nitrogen to prevent any possible carbamate formation). This was converted to its hydrochloride salt by applying the method described under General method for the hydrochloride salt formations. Yield: 71.8 mg (quantitative) product 55 as a beige solid. ¹H-NMR (D₂O): 2.95 (t, ArCH₂), 3.10 (t, CH₂NH₃_{⁺}), 3.48 (s, HCC), 3.79 (s, 2×MeO), 6.85 (s, 2 arom. H).

N-Trifluoroacetyl-2,6-dimethoxy-4-(trimethylsilylethynyl)amphetamine, 54. To a mixture of 1.84 g (4.96 mmol) 44, 40 mg (0.2 mmol) CuI and 70 mg (0.1 mmol) PdCl₂(PPh₃)₂ in 20 mL THF anhydr. under nitrogen was added 1.76 mL (19.8 mmol) triethylamine in one portion. After stirring for 1 min, 1.44 mL (10.4 mmol) ethynyltrimethylsilane in 2 mL THF anhydr. was added during 5 min (generally, slow addition prevents the formation of diynes). This reaction was essentially slow and thus, over the course of 5 days, there was added another 0.5 equals of the above amounts of palladium catalyst and alkyne on day 2, 3, 4 and 5. After stirring for a total of 7 days the solvent was evaporated. The residue was redissolved in a minimal amount of DCM and pre-coated on silica gel (8 g) and then the DCM was evaporated. The coated product was plugged on a silica gel column (d: 4 cm, h: 16 cm) preconditioned with hexane/ethyl acetate 95:5 and eluted (95:5 to 9:1) to get 390 mg (20.3%) product 54 as a beige-brownish solid. ¹H-NMR (CDCl₃; a superimposition of either rotamers and/or cis/trans isomers of the amide function was observed. LCMS: eluted as a single compound): 0.29 (m, Me₃Si), 1.22 (d (superimposed), MeCH), 2.87 (m, ArCH₂), 3.86 (m, 2×MeO), 4.06 (m, CHNH), 6.70/6.86 (s, 2 arom. H), 7.17/7.26 (bs, NH).

2,6-Dimethoxy-4-ethynylamphetamine hydrochloride (ψ-DOYN), 56. To a solution of 387 mg (1.00 mmol) trimethylsilylethynyl intermediate 54 in 80 mL MeOH were added 2 mL K₂CO₃ 2M aq. and after stirring for 75 min LCMS indicated complete TMS removal. An aq. 2M NaOH solution (25 mL; Note: much more was needed than for the preparation of 55) was added and stirring was continued for 5 h. The MeOH was removed by using a rotary evaporator (40° C.), and the residue was taken up with 50 mL MTBE and washed with 3×30 mL water, dried over Na₂SO₄ whereby 2 g activated charcoal pellets were added prior filtering off, and the filtrate was concentrated in vacuo to get the free base of 56 (181 mg, brownish oil; note: purging of the rotary evaporator was performed with nitrogen to prevent any possible carbamate formation). This was converted to its hydrochloride salt by applying the method described under General method for the hydrochloride salt formations. Yield: 213 mg (quantitative) product 56 as a beige solid. ¹H-NMR (D₂O): 1.23 (d, MeCH), 2.88 (m, ArCH₂), 3.47 (s (superimposed), HCC), 3.50 (m (superimposed), CHNH₃_{⁺}), 3.77 (s, 2×MeO), 6.83 (s, 2 arom. H).

### REFERENCES

1. Bogenschutz M P (2013). Studying the effects of classic hallucinogens in the treatment of alcoholism: rationale, methodology, and current research with psilocybin. Curr Drug Abuse Rev 6: 17-29.
2. 2. Bogenschutz M P, Forcehimes A A, Pommy J A, Wilcox C E, Barbosa P C, & Strassman R J (2015). Psilocybin-assisted treatment for alcohol dependence: a proof-of-concept study. J Psychopharmacol 29: 289-299.
3. Braden M R, & Nichols D E (2007). Assessment of the roles of serines 5.43(239) and 5.46(242) for binding and potency of agonist ligands at the human serotonin 5-HT2A receptor. Mol Pharmacol 72: 1200-1209.
4. Byrne P A, & Gilheany D G (2012). Unequivocal experimental evidence for a unified lithium salt-free Wittig reaction mechanism for all phosphonium Ylide types: reactions with beta-heteroatom-substituted aldehydes are consistently selective for cis-oxaphosphetane-derived products. J Am Chem Soc 134: 9225-9239.
5. Carhart-Harris R, Giribaldi B, Watts R, Baker-Jones M, Murphy-Beiner A, Murphy R, Martell J, Blemings A, Erritzoe D, & Nutt D J (2021). Trial of psilocybin versus escitalopram for depression. N Engl J Med 384: 1402-1411.
6. Carhart-Harris R L, Bolstridge M, Rucker J, Day C M, Erritzoe D, Kaelen M, Bloomfield M, Rickard J A, Forbes B, Feilding A, Taylor D, Pilling S, Curran V H, & Nutt D J (2016a). Psilocybin with psychological support for treatment-resistant depression: an open-label feasibility study. Lancet Psychiatry 3: 619-627.
7. Carhart-Harris R L, Kaelen M, Bolstridge M, Williams T M, Williams L T, Underwood R, Feilding A, & Nutt D J (2016b). The paradoxical psychological effects of lysergic acid diethylamide (LSD). Psychol Med 46: 1379-1390.
8. Chambers J J, Kurrasch-Orbaugh D M, & Nichols D E (2002). Translocation of the 5-alkoxy substituent of 2,5-dialkoxyarylalkylamines to the 6-position: effects on 5-HT(2A/2C) receptor affinity. Bioorg Med Chem Lett 12: 1997-1999.
9. Davis A K, Barrett F S, May D G, Cosimano M P, Sepeda N D, Johnson M W, Finan P H, & Griffiths R R (2021). Effects of psilocybin-assisted therapy on major depressive disorder: a randomized clinical trial. JAMA Psychiatry 78: 481-489.
10. Dolder P C, Schmid Y, Mueller F, Borgwardt S, & Liechti M E (2016). LSD acutely impairs fear recognition and enhances emotional empathy and sociality.

Neuropsychopharmacology 41: 2638-2646.
11. Dolder P C, Schmid Y, Steuer A E, Kraemer T, Rentsch K M, Hammann F, & Liechti M E (2017). Pharmacokinetics and pharmacodynamics of lysergic acid diethylamide in healthy subjects. Clin Pharmacokinetics 56: 1219-1230.
12. Dominguez-Clave E, Soler J, Elices M, Pascual J C, Alvarez E, de la Fuente Revenga M, Friedlander P, Feilding A, & Riba J (2016). Ayahuasca: Pharmacology, neuroscience and therapeutic potential. Brain Res Bull 126: 89-101.
13. Dos Santos R G, Osorio F L, Crippa J A, Riba J, Zuardi A W, & Hallak J E (2016). Antidepressive, anxiolytic, and antiaddictive effects of ayahuasca, psilocybin and lysergic acid diethylamide (LSD): a systematic review of clinical trials published in the last 25 years. Ther Adv Psychopharmacol 6: 193-213.
14. Gasser P, Holstein D, Michel Y, Doblin R, Yazar-Klosinski B, Passie T, & Brenneisen R (2014). Safety and efficacy of lysergic acid diethylamide-assisted psychotherapy for anxiety associated with life-threatening diseases. J Nerv Ment Dis 202: 513-520.
15. Gasser P, Kirchner K, & Passie T (2015). LSD-assisted psychotherapy for anxiety associated with a life-threatening disease: a qualitative study of acute and sustained subjective effects. J Psychopharmacol 29: 57-68.
16. Glennon R A, Raghupathi R, Bartyzel P, Teitler M, & Leonhardt S (1992). Binding of phenylalkylamine derivatives at 5-HT1C and 5-HT2 serotonin receptors: evidence for a lack of selectivity. J Med Chem 35: 734-740.
17. Glennon R A, Rosecrans J A, & Young R (1981). Behavioral properties of psychoactive phenylisopropylamines in rats. Eur J Pharmacol 76: 353-360.
18. Griffiths R, Richards W, Johnson M, McCann U, & Jesse R (2008). Mystical-type experiences occasioned by psilocybin mediate the attribution of personal meaning and spiritual significance 14 months later. J Psychopharmacol 22: 621-632.
19. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.
20. Grob C S, Danforth A L, Chopra G S, Hagerty M, McKay C R, Halberstadt A L, & Greer G R (2011). Pilot study of psilocybin treatment for anxiety in patients with advanced-stage cancer. Arch Gen Psychiatry 68: 71-78.
21. Hathaway B A, Taylor B E, & Wittenborn J S (1998). Selective preparation of 4-(bromo or iodo)-2,5-dimethoxybenzonitrile and 2-(bromo or iodo)-3,6-dimethoxybenzonitrile from 2,5-dimethoxybenzaldehyde. Synthetic Communications 28: 4629-4637.
22. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.
23. Holze F, Vizeli P, Ley L, Muller F, Dolder P, Stocker M, Duthaler U, Varghese N, Eckert A, Borgwardt S, & Liechti M E (2021). Acute dose-dependent effects of lysergic acid diethylamide in a double-blind placebo-controlled study in healthy subjects.

Neuropsychopharmacology 46: 537-544.
24. Hysek C M, Simmler L D, Nicola V, Vischer N, Donzelli M, Krahenbuhl S, Grouzmann E, Hoener M C, & Liechti M E (2012). Duloxetine inhibits effects of MDMA (“ecstasy”) in vitro and in humans in a randomized placebo-controlled laboratory study. PLoS One 7: e36476.
25. Ichikawa J, & Meltzer H Y (2000). The effect of serotonin(1A) receptor agonism on antipsychotic drug-induced dopamine release in rat striatum and nucleus accumbens. Brain Res 858: 252-263.
26. Janowsky A, Eshleman A J, Johnson R A, Wolfrum K M, Hinrichs D J, Yang J, Zabriskie T M, Smilkstein M J, & Riscoe M K (2014). Mefloquine and psychotomimetics share neurotransmitter receptor and transporter interactions in vitro. Psychopharmacology (Berl) 231: 2771-2783.
27. Johnson M P, Mathis C A, Shulgin A T, Hoffman A J, & Nichols D E (1990). [1251]-2-(2,5-dimethoxy-4-iodophenyl)aminoethane ([125I]-2C-I) as a label for the 5-HT2 receptor in rat frontal cortex. Pharmacol Biochem Behav 35: 211-217.
28. Johnson M W, Garcia-Romeu A, Cosimano M P, & Griffiths R R (2014). Pilot study of the 5-HT2A R agonist psilocybin in the treatment of tobacco addiction. J Psychopharmacol 28: 983-992.
29. Johnson M W, Garcia-Romeu A, & Griffiths R R (2016). Long-term follow-up of psilocybin-facilitated smoking cessation. Am J Drug Alcohol Abuse 43: 55-60.
30. Krebs T S, & Johansen P O (2012). Lysergic acid diethylamide (LSD) for alcoholism: meta-analysis of randomized controlled trials. J Psychopharmacol 26: 994-1002.
31. Liechti M E (2017). Modern clinical research on LSD. Neuropsychopharmacology 42: 2114-2127.
32. Luethi D, & Liechti M E (2018). Monoamine transporter and receptor interaction profiles in vitro predict reported human doses of novel psychoactive stimulants and psychedelics. Int J Neuropsychopharmacol 21: 926-931.
33. Madsen M K, Fisher P M, Burmester D, Dyssegaard A, Stenbaek D S, Kristiansen S, Johansen S S, Lehel S, Linnet K, Svarer C, Erritzoe D, Ozenne B, & Knudsen G M (2019). Correction: Psychedelic effects of psilocybin correlate with serotonin 2A receptor occupancy and plasma psilocin levels. Neuropsychopharmacology 44: 1328-1334.
34. Märcher-Rorsted E, Nykodermova J, & Kristensen J L (2021). An improved, scalable synthesis of the selective serotonin 2A receptor agonist 25C N-NBOH. SynOpen 5: 158-160.
35. Marona-Lewicka D, Thisted R A, & Nichols D E (2005). Distinct temporal phases in the behavioral pharmacology of LSD: dopamine D2 receptor-mediated effects in the rat and implications for psychosis. Psychopharmacology (Berl) 180: 427-435.
36. Nelson D L, Lucaites V L, Wainscott D B, & Glennon R A (1999). Comparisons of hallucinogenic phenylisopropylamine binding affinities at cloned human 5-HT2A, -HT(2B) and 5-HT2C receptors. Naunyn Schmiedebergs Arch Pharmacol 359: 1-6.
37. Nichols D E, Frescas S, Marona-Lewicka D, Huang X, Roth B L, Gudelsky G A, & Nash J F (1994). 1-(2,5-Dimethoxy-4-(trifluoromethyl)phenyl)-2-aminopropane: a potent serotonin 5-HT2A/2C agonist. J Med Chem 37: 4346-4351.
38. Oberlender R, Ramachandran P V, Johnson M P, Huang X, & Nichols D E (1995). Effect of a chiral 4-alkyl substituent in hallucinogenic amphetamines. J Med Chem 38: 3593-3601.
39. Palhano-Fontes F, Barreto D, Onias H, Andrade K C, Novaes M M, Pessoa J A, Mota-Rolim S A, Osorio F L, Sanches R, Dos Santos R G, Tofoli L F, de Oliveira Silveira G, Yonamine M, Riba J, Santos F R, Silva-Junior A A, Alchieri J C, Galvao-Coelho N L, Lobao-Soares B, Hallak J E C, Arcoverde E, Maia-de-Oliveira J P, & Araujo D B (2019). Rapid antidepressant effects of the psychedelic ayahuasca in treatment-resistant depression: a randomized placebo-controlled trial. Psychol Med 49: 655-663.
40. Parker M A, Kurrasch D M, & Nichols D E (2008). The role of lipophilicity in determining binding affinity and functional activity for 5-HT2A receptor ligands. Bioorg Med Chem 16: 4661-4669.
41. Passie T, Halpern J H, Stichtenoth D O, Emrich H M, & Hintzen A (2008). The pharmacology of lysergic acid diethylamide: a review. CNS Neurosci Ther 14: 295-314.
42. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.
43. Roseman L, Nutt D J, & Carhart-Harris R L (2017). Quality of acute psychedelic experience predicts therapeutic efficacy of psilocybin for treatment-resistant depression. Front Pharmacol 8: 974.
44. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.
45. Sanches R F, de Lima Osorio F, Dos Santos R G, Macedo L R, Maia-de-Oliveira J P, Wichert-Ana L, de Araujo D B, Riba J, Crippa J A, & Hallak J E (2016). Antidepressant Effects of a Single Dose of Ayahuasca in Patients With Recurrent Depression: A SPECT Study. J Clin Psychopharmacol 36: 77-81.
46. Schlosser M, & Zimmermann M (1969). Fluor-olefine durch Fluormethylenierung von Carbonylverbindungen. Synthesis 2: 75-76.
47. Schmid Y, Enzler F, Gasser P, Grouzmann E, Preller K H, Vollenweider F X, Brenneisen R, Mueller F, Borgwardt S, & Liechti M E (2015). Acute effects of lysergic acid diethylamide in healthy subjects. Biol Psychiatry 78: 544-553.
48. Schmid Y, Gasser P, Oehen P, & Liechti M E (2021). Acute subjective effects in LSD- and MDMA-assisted psychotherapy. J Psychopharmacol 35: 362-374.
49. Schmid Y, & Liechti M E (2018). Long-lasting subjective effects of LSD in normal subjects. Psychopharmacology (Berl) 235: 535-545.
50. Shulgin A, & Shulgin A (1991) PiHKAL: a chemical love story. 1 edn.
51. Snyder S H, Faillace L, & Hollister L (1967). 2,5-dimethoxy-4-methyl-amphetamine (STP): a new hallucinogenic drug. Science 158: 669-670.
52. Trachsel D (2002). Synthese von neuen (Phenylalkyl)aminen zur Untersuchung von Struktur-Aktivitatsbeziehungen, Mitteilung 1: Mescalin Derivative. Helvetica Chimica Acta 85: 3019-3026.
53. Trachsel D (2003). Synthesis of novel (phenethylalkyl)amines for the investigation of struture-activity relationships, Part 3. Helv Chim Acta 86: 2754-2759.
54. Trachsel D, Lehmann D, & Enzensperger C (2013) Phenethylemine: Von der Struktur zur Funktion. 1 edn.: Solothurn: Nachtschatten Verlag.
55. Vollenweider F X, Vontobel P, Hell D, & Leenders K L (1999). 5-H T modulation of dopamine release in basal ganglia in psilocybin-induced psychosis in man—a PET study with [11C]raclopride. Neuropsychopharmacology 20: 424-433.
56. Zheng J, Cai J, Lin J H, Guo Y, & Xiao J C (2013). Synthesis and decarboxylative Wittig reaction of difluoromethylene phosphobetaine. Chem Commun (Camb) 49: 7513-7515.

What is claimed is:

1. A composition comprising a compound represented by FIG. 1A-1E,
characterized in that R_{□1} and R_{□2} is, independently and in any combination, chosen from the group consisting of hydrogen, deuteron, methyl, ethyl, deuterated methyl (D₁-D₃), or deuterated ethyl (D₁-D₅), and
further characterized in that R1, R2 and R3 are, independently and in any combination, chosen from the group consisting of hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
any halogen or a nitrogen-containing substituent such as CN or NO₂; and
further characterized in that R4 is chosen from the group consisting of hydrogen, deuteron, or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN; and
further characterized in that R5 is chosen from the group consisting of fluorine, chlorine, bromine, or iodine; or
SF₅; or
C₂-C₅ branched or unbranched alkyl with the alkyl optionally and independently substituted with F₁-F₁₁ fluorine and/or D₁-D₁₁ deuteron substituents or C₁ alkyl substituted with F₁-F₃ and/or D₁-D₃ deuteron substituents; or
C₃-C₆ cycloalkyl optionally and independently substituted with one or more substituents such as F₁-F₁₅ fluorine and/or D₁-D₁₅ deuteron and/or C₁-C₅ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₅ alkyl, a nitrogen-containing substituent such as CN or NO₂; or
C₂-C₅ branched or unbranched alkoxy with the alkoxy optionally and independently substituted with F₁-F₁₁ fluorine and/or D₁-D₁₁ substituents, or C₁ alkoxy substituted with F₁-F₃ and/or D₁-D₃ deuteron substituents; or
C₃-C₆ cycloalkyloxy optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkoxy optionally and independently substituted with one or more substituents such as F₁-F₁₅ fluorine and/or D₁-D₁₅ and/or C₁-C₅ alkyl; or
C₂-C₅ branched or unbranched alkenyloxy with E or Z vinylic, cis or trans allylic, E or Z allylic or other double bond position in relation to the attached ether function, where any of the carbons of the branched or unbranched alkenyloxy substituent is optionally substituted independently with one or more C₁-C₅ alkyl, and/or with F₁-F₉ fluorine and/or with D₁-D₉ deuteron substituents; or
C₃-C₅ branched or unbranched alkynyloxy, where any of the carbons of the alkynyloxy substituent is optionally substituted independently with one or more C₁-C₅ alkyl, and/or with F₁-F₉ fluorine and/or with D₁-D₉ deuteron substituents; or
C₃-C₅ branched or unbranched alkylthio with the alkylthio group optionally and independently substituted with F₁-F₁₁ fluorine and/or D₁-D₁₁ substituents, or C₁-C₂ alkylthio substituted with F₁-F₅ and/or D₁-D₅ deuteron substituents; or
C₃-C₆ cycloalkylthio optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkylthio optionally and independently substituted with one or more substituents such as F₁-F₁₅ fluorine and/or D₁-D₁₅ and/or C₁-C₅ alkyl; or
C₂-C₅ branched or unbranched alkenylthio with E or Z vinylic, cis or trans allylic, E or Z allylic or other double bond position in relation to the attached thioether function, where any of the carbons of the branched or unbranched alkenylthio substituent is optionally substituted independently with one or more C₁-C₅ alkyl, and/or with F₁-F₉ fluorine and/or with D₁-D₉ deuteron substituents; or
C₃-C₅ branched or unbranched alkynylthio, where any of the carbons of the alkynylthio substituent is optionally substituted independently with one or more C₁-C₅ alkyl, and/or with F₁-F₉ fluorine and/or with D₁-D₉ deuteron substituents,
and further characterized in that any non-protic hydrogen can be replaced by a deuteron or a fluorine in any combination.

2. The composition of claim 1, wherein said compound is a free base.

3. The composition of claim 1, wherein said compound is a salt thereof.

4. The composition of claim 3, wherein said compound is a hydrochloride salt or a fumarate salt thereof.

5. The composition of claim 4, wherein said compound is a pharmacologically acceptable acid addition salt thereof.

6. The composition of claim 1, wherein said compound is chosen from the group consisting of a racemate, a single enantiomer, a diastereomer, and a mixture of enantiomers or diastereomers in any ratio, a single and a mixture of E or Z configurational isomer in any ratio, a single and a mixture of cis or trans configurational isomer in any ratio, or combinations thereof.

7. The composition of claim 1, wherein said compound includes a prodrug.

8. The composition of claim 1, wherein said prodrug is chosen from the group consisting of amide, carbamate, ureate, N-Mannich base, amino sugars, mines (Schiff bases), examines, enaminones, and THTT.

9. A method of changing neurotransmission, including the steps of:
administering a pharmaceutically effective amount of composition to a mammal of a compound represented by FIG. 1A-1E,
characterized in that R_{□1} and R_{□2} are, independently and in any combination, chosen from the group consisting of hydrogen, deuteron, methyl, ethyl, deuterated methyl (D₁-D₃), or deuterated ethyl (D₁-D₅), and
further characterized in that R1, R2 and R3 are, independently and in any combination, chosen from the group consisting of hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN or NO₂; and
further characterized in that R4 is chosen from the group consisting of hydrogen, deuteron, or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN; and
further characterized in that R5 is chosen from the group consisting of fluorine, chlorine, bromine, and iodine; or
SF₅; or
C₂-C₅ branched or unbranched alkyl with the alkyl optionally and independently substituted with F₁-F₁₁ fluorine and/or D₁-D₁₁ deuteron substituents or C₁ alkyl substituted with F₁-F₃ and/or D₁-D₃ deuteron substituents; or
C₃-C₆ cycloalkyl optionally and independently substituted with one or more substituents such as F₁-F₁₅ fluorine and/or D₁-D₁₅ deuteron and/or C₁-C₅ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₅ alkyl; or
a nitrogen-containing substituent such as CN or NO₂; or
C₂-C₅ branched or unbranched alkoxy with the alkoxy optionally and independently substituted with F₁-F₁₁ fluorine and/or D₁-D₁₁ substituents, or C₁ alkoxy substituted with F₁-F₃ and/or D₁-D₃ deuteron substituents; or
C₃-C₆ cycloalkyloxy optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkoxy optionally and independently substituted with one or more substituents such as F₁-F₁₅ fluorine and/or D₁-D₁₅ and/or C₁-C₅ alkyl; or
C₂-C₅ branched or unbranched alkenyloxy with E or Z vinylic, cis or trans allylic, E or Z allylic or other double bond position in relation to the attached ether function, where any of the carbons of the branched or unbranched alkenyloxy substituent is optionally substituted independently with one or more C₁-C₅ alkyl, and/or with F₁-F₉ fluorine and/or with D₁-D₉ deuteron substituents; or
C₃-C₅ branched or unbranched alkynyloxy, where any of the carbons of the alkynyloxy substituent is optionally substituted independently with one or more C₁-C₅ alkyl, and/or with F₁-F₉ fluorine and/or with D₁-D₉ deuteron substituents; or
C₃-C₅ branched or unbranched alkylthio with the alkylthio group optionally and independently substituted with F₁-F₁₁ fluorine and/or D₁-D₁₁ substituents, or C₁-C₂ alkylthio substituted with F₁-F₅ and/or D₁-D₅ deuteron substituents; or
C₃-C₆ cycloalkylthio optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkylthio optionally and independently substituted with one or more substituents such as F₁-F₁₅ fluorine and/or D₁-D₁₅ and/or C₁-C₅ alkyl; or
C₂-C₅ branched or unbranched alkenylthio with E or Z vinylic, cis or trans allylic, E or Z allylic or other double bond position in relation to the attached thioether function, where any of the carbons of the branched or unbranched alkenylthio substituent is optionally substituted independently with one or more C₁-C₅ alkyl, and/or with F₁-F₉ fluorine and/or with D₁-D₉ deuteron substituents; or
C₃-C₅ branched or unbranched alkynylthio, where any of the carbons of the alkynylthio substituent is optionally substituted independently with one or more C₁-C₅ alkyl, and/or with F₁-F₉ fluorine and/or with D₁-D₉ deuteron substituents,
and further characterized in that any non-protic hydrogen can be replaced by a deuteron or a fluorine in any combination;
increasing serotonin 5-HT2A and 5-HT2C receptor interaction in the mammal; and
inducing psychoactive effects.

10. The method of claim 9, wherein the compound is chosen from the group consisting of a racemate, a single enantiomer, a diastereomer, and a mixture of enantiomers or diastereomers in any ratio, a single and a mixture of E or Z configurational isomer in any ratio, a single and a mixture of cis or trans configurational isomer in any ratio, or combinations thereof.

11. The method of claim 9, wherein the psychoactive effects include psychedelic or empathogenic effects having intensity, effect quality, or duration of effect in a mammal in comparison to that of DOM, mescaline, LSD, or psilocybin.

12. The method of claim 9, wherein the compound is administered to mammals for substance-assisted psychotherapy.

13. The method of claim 9, wherein the compound is administered to allow for changing dose potency in comparison to DOM.

14. The method of claim 9, wherein the compound is administered to allow for tailoring and treatment individualization to the mammal's therapeutic need.

15. The method of claim 9, wherein the mammal is a human.

16. A method of treating a patient having adverse reactions to psychedelics, including the steps of:
administering a pharmaceutically effective amount of composition to the patient of a compound represented by FIG. 1A-1E,
characterized in that R_{□1} and R_{□2} are, independently and in any combination, chosen from the group consisting of hydrogen, deuteron, methyl, ethyl, deuterated methyl (D₁-D₃), or deuterated ethyl (D₁-D₅), and
further characterized in that R1, R2 and R3 are, independently and in any combination, chosen from the group consisting of hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN or NO₂; and
further characterized in that R4 is chosen from the group consisting of hydrogen, deuteron, or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN; and
further characterized in that R5 is chosen from the group consisting of fluorine, chlorine, bromine, and iodine; or
SF₅; or
C₂-C₅ branched or unbranched alkyl with the alkyl optionally and independently substituted with F₁-F₁₁ fluorine and/or D₁-D₁₁ deuteron substituents or C₁ alkyl substituted with F₁-F₃ and/or D₁-D₃ deuteron substituents; or
C₃-C₆ cycloalkyl optionally and independently substituted with one or more substituents such as F₁-F₁₅ fluorine and/or D₁-D₁₅ deuteron and/or C₁-C₅ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₅ alkyl; or
a nitrogen-containing substituent such as CN or NO₂; or
C₂-C₅ branched or unbranched alkoxy with the alkoxy optionally and independently substituted with F₁-F₁₁ fluorine and/or D₁-D₁₁ substituents, or C₁ alkoxy substituted with F₁-F₃ and/or D₁-D₃ deuteron substituents; or
C₃-C₆ cycloalkyloxy optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkoxy optionally and independently substituted with one or more substituents such as F₁-F₁₅ fluorine and/or D₁-D₁₅ and/or C₁-C₅ alkyl; or
C₂-C₅ branched or unbranched alkenyloxy with E or Z vinylic, cis or trans allylic, E or Z allylic or other double bond position in relation to the attached ether function, where any of the carbons of the branched or unbranched alkenyloxy substituent is optionally substituted independently with one or more C₁-C₅ alkyl, and/or with F₁-F₉ fluorine and/or with D₁-D₉ deuteron substituents; or
C₃-C₅ branched or unbranched alkynyloxy, where any of the carbons of the alkynyloxy substituent is optionally substituted independently with one or more C₁-C₅ alkyl, and/or with F₁-F₉ fluorine and/or with D₁-D₉ deuteron substituents; or
C₃-C₅ branched or unbranched alkylthio with the alkylthio group optionally and independently substituted with F₁-F₁₁ fluorine and/or D₁-D₁₁ substituents, or C₁-C₂ alkylthio substituted with F₁-F₅ and/or D₁-D₅ deuteron substituents; or
C₃-C₆ cycloalkylthio optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkylthio optionally and independently substituted with one or more substituents such as F₁-F₁₅ fluorine and/or D₁-D₁₅ and/or C₁-C₅ alkyl; or
C₂-C₅ branched or unbranched alkenylthio with E or Z vinylic, cis or trans allylic, E or Z allylic or other double bond position in relation to the attached thioether function, where any of the carbons of the branched or unbranched alkenylthio substituent is optionally substituted independently with one or more C₁-C₅ alkyl, and/or with F₁-F₉ fluorine and/or with D₁-D₉ deuteron substituents; or
C₃-C₅ branched or unbranched alkynylthio, where any of the carbons of the alkynylthio substituent is optionally substituted independently with one or more C₁-C₅ alkyl, and/or with F₁-F₉ fluorine and/or with D₁-D₉ deuteron substituents, and further characterized in that any non-protic hydrogen can be replaced by a deuteron or a fluorine in any combination; and
avoiding adverse effects present with psychedelics.

17. The method of claim 16, wherein the adverse effects are chosen from the group consisting of less anxiety, less cardio-stimulant effects, less thermogenesis, less adverse effects, less nausea, and combinations thereof.

18. The method of claim 17, further including the step of providing more positive effects than other psychedelics.

19. The method of claim 18, wherein the positive effects are chosen from the group consisting of more overall positive effects, more or less perceptual effects, more emotional effects, and combinations thereof.

20. The method of claim 16, further including the step of providing a shorter duration of action of the DOM molecule than with other psychedelics.

21. A method of changing neurotransmission of an individual, including the steps of:
administering a pharmaceutically effective amount of composition to the individual of a compound represented by FIG. 1A-1E,
characterized in that R_{□1} and R_{□2} are, independently and in any combination, chosen from the group consisting of hydrogen, deuteron, methyl, ethyl, deuterated methyl (D₁-D₃), or deuterated ethyl (D₁-D₅), and
further characterized in that R1, R2 and R3 are, independently and in any combination, chosen from the group consisting of hydrogen, deuteron, and/or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN or NO₂; and
further characterized in that R4 is chosen from the group consisting of hydrogen, deuteron, or fluorine; or
C₁-C₃ branched or unbranched alkyl with the alkyl optionally and independently substituted with F₁-F₇ fluorine and/or D₁-D₇ deuteron substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₂ alkyl; or
C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, any halogen or a nitrogen-containing substituent such as CN; and
further characterized in that R5 is chosen from the group consisting of fluorine, chlorine, bromine, and iodine; or
SF₅; or
C₂-C₅ branched or unbranched alkyl with the alkyl optionally and independently substituted with F₁-F₁₁ fluorine and/or D₁-D₁₁ deuteron substituents or C₁ alkyl substituted with F₁-F₃ and/or D₁-D₃ deuteron substituents; or
C₃-C₆ cycloalkyl optionally and independently substituted with one or more substituents such as F₁-F₁₅ fluorine and/or D₁-D₁₅ deuteron and/or C₁-C₅ alkyl; or
(C₃-C₆ cycloalkyl)-C₁-C₂ branched or unbranched alkyl optionally and independently substituted with one or more substituents such as F₁-F₁₅ fluorine and/or D₁-D₁₅ deuteron and/or C₁-C₅ alkyl; or
a nitrogen-containing substituent such as CN or NO₂; or
C₂-C₅ branched or unbranched alkoxy with the alkoxy optionally and independently substituted with F₁-F₁₁ fluorine and/or D₁-D₁₁ substituents, or C₁ alkoxy substituted with F₁-F₃ and/or D₁-D₃ deuteron substituents; or
C₃-C₆ cycloalkyloxy optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkoxy optionally and independently substituted with one or more substituents such as F₁-F₁₅ fluorine and/or D₁-D₁₅ and/or C₁-C₅ alkyl; or
C₂-C₅ branched or unbranched alkenyloxy with E or Z vinylic, cis or trans allylic, E or Z allylic or other double bond position in relation to the attached ether function, where any of the carbons of the branched or unbranched alkenyloxy substituent is optionally substituted independently with one or more C₁-C₅ alkyl, and/or with F₁-F₉ fluorine and/or with D₁-D₉ deuteron substituents; or
C₃-C₅ branched or unbranched alkynyloxy, where any of the carbons of the alkynyloxy substituent is optionally substituted independently with one or more C₁-C₅ alkyl, and/or with F₁-F₉ fluorine and/or with D₁-D₉ deuteron substituents; or
C₃-C₅ branched or unbranched alkylthio with the alkylthio group optionally and independently substituted with F₁-F₁₁ fluorine and/or D₁-D₁₁ substituents, or C₁-C₂ alkylthio substituted with F₁-F₅ and/or D₁-D₅ deuteron substituents; or
C₃-C₆ cycloalkylthio optionally and independently substituted with one or more substituents such as F₁-F₁₁ fluorine and/or D₁-D₁₁ substituents and/or C₁-C₅ alkyl; or
(C₃-C₆ cycloalkyl)-C₁-C₂ branched or unbranched alkylthio optionally and independently substituted with one or more substituents such as F₁-F₁₅ fluorine and/or D₁-D₁₅ and/or C₁-C₅ alkyl; or
C₂-C₅ branched or unbranched alkenylthio with E or Z vinylic, cis or trans allylic, E or Z allylic or other double bond position in relation to the attached thioether function, where any of the carbons of the branched or unbranched alkenylthio substituent is optionally substituted independently with one or more C₁-C₅ alkyl, and/or with F₁-F₉ fluorine and/or with D₁-D₉ deuteron substituents; or
C₃-C₅ branched or unbranched alkynylthio, where any of the carbons of the alkynylthio substituent is optionally substituted independently with one or more C₁-C₅ alkyl, and/or with F₁-F₉ fluorine and/or with D₁-D₉ deuteron substituents, and further characterized in that any non-protic hydrogen can be replaced by a deuteron or a fluorine in any combination; and
changing neurotransmission in the individual.
